(19) United States
(12) Patent Application Publication
(10) Pub. No.: US 2022/0157414 A1
(43) Pub. Date: May 19, 2022

(54) METHOD AND SYSTEM FOR FACILITATING OPTIMIZATION OF A CLUSTER COMPUTING NETWORK FOR SEQUENCING DATA ANALYSIS USING ADAPTIVE DATA PARALLELIZATION, AND NON-TRANSITORY STORAGE MEDIUM

(71) Applicant: ATGENOMIX INC., Taipei City (TW)

(72) Inventors: MING-TAI CHANG, New Taipei City (TW); CHUNG-TSAI SU, New Taipei City (TW); YUN-LUNG LI, Taipei City (TW); WEN-CHIEN WENG, Taipei City (TW)

(21) Appl. No.: 17/098,477

(22) Filed: Nov. 16, 2020

(51) Int. Cl. G16H 10/40; G06F 9/50; H04L 12/24

(52) U.S. Cl. CPC G16H 10/40; H04L 41/0826; G06F 9/5083

(57) ABSTRACT

A method for facilitating optimization of a cluster computing network for sequencing data analysis using adaptive data parallelization is provided. The method comprises the following steps. (a) A data parallelization configuration is determined, based on sequencing data and a pipeline selection, wherein the data parallelization configuration includes partition indication data indicating at least one biological information unit based on which of the sequencing data is to be partitioned. (b) At least one recommendation list is determined, based on the data parallelization configuration and a computing resource list for the cluster computing network, wherein the at least one recommendation list is for a computing device to produce at least one resource allocation selection from the at least one recommendation list so that the cluster computing network can perform the sequencing data analysis on the sequencing data, according to the at least one resource allocation selection and the data parallelization configuration.

### BACKGROUND OF THE INVENTION

### 1. Field of the Invention

The present disclosure relates to sequencing data analysis, and in particular to a method and a system for facilitating optimization of a cluster computing network for sequencing data analysis using adaptive data parallelization, and a non-transitory storage medium.

### 2. Description of the Related Art

Whole genome sequencing, such as Next-generation sequencing (NGS), is progressively more applied to biomedical research, clinical, and personalized medicine applications to identify disease- and/or drug-associated genetic variants to advance precision medicine. The impact of NGS technologies in revolutionizing the biological and clinical sciences has been unprecedented (Goodwin, S. et al, Nature Reviews Genetics 17, 333-351 (2016); Ashley, E., et al, Nature Reviews Genetics 17, 507-522 (2016)).

Since there are over three billion base pairs (sites) on a human genome, sequencing a whole genome generates more than 100 gigabytes of data in FASTQ, BAM (the binary version of sequence alignment/map) and VCF (Variant Call Format) file formats. Compounded by sharply falling sequencing costs, this exponential growth in NGS data generation has created a computational and bioinformatics bottleneck in which current approaches can take over a week to complete sequence data analysis and interpretation. These challenges have created the need for a pipeline that would both streamline the bioinformatics analysis required to utilize these tools and dramatically reduce the turnaround time.

Referring to FIG. 1, post-sequencing DNA analysis typically includes read mapping and variant calling, wherein annotation is optional. The analysis is very time-consuming computationally, especially for whole genome sequencing. With the ever increasing rate at which next-generation sequencing (NGS) data is generated, it is important to improve the data processing and analysis workflow.

A number of tools such as HugeSeq [Lam HYK. et al Nature Biotechnology. 2012 Mar.;30(3):226-229], MegaSeq [Puckelwartz MJ. et al Bioinformatics. 2014 Jun.;30(11):1508-1513], Churchill, an HPC cluster-based solution [Kelly BJ. et al Genome biology. 2015 Jan.;16(1)] and Halvade, a Hadoop MapReduce solution, [Decap D. et al Bioinformatics. 2015 Mar.;31(15):2482-2488] have been introduced to improve the data processing and analysis workflow.

Halvade provides a parallel, multi-node framework for read alignment and variant calling that relies on the MapReduce programming model. Read alignment is then performed during the mapping phase, while variant calling is handled in the reduction phase. A variant calling pipeline based on the GATK Best Practices recommendations (BWA, Picard and GATK) has been implemented in Halvade and shown to significantly reduce the runtime. Halvade uses a fixed-length partitioning method with a certain degree of overlap.

Unfortunately, the fixed-length partitioning method may result in a loss of biologically significant information since an association signal may be split up by a fixed-length partition. FIG. 2 illustrates a genome with a gene body including structural variations, represented by SVar, wherein the structural variations SVar, correspondingly represented by bolded line segments, are distributed in the sequencing data of the genome. As illustrated in FIG. 2, after fixed-length partitioning, the structural variations are split into two partitions (e.g., partitions 2 and 3) or some of them are even truncated, thus leading to loss of biologically significant information.

### BRIEF SUMMARY OF THE INVENTION

An objective of the present disclosure is to provide technology for facilitating optimization of a cluster computing network for sequencing data analysis using adaptive data parallelization. The technology facilitates that the sequencing data analysis can be performed by using recommended computing resource and adaptive data parallelization, without biological meaning loss. As a result, the sequencing data analysis can be achieved with efficiency and cost-effectiveness and without biological meaning loss.

The present disclosure provides a method for facilitating optimization of a cluster computing network for sequencing data analysis using adaptive data parallelization. The method comprises the following steps. (a) A data parallelization configuration is determined, based on sequencing data and a pipeline selection, by one or more processing units, wherein the data parallelization configuration includes partition indication data indicating at least one biological information unit according to which of the sequencing data is to be partitioned. (b) At least one recommendation list is determined, based on the data parallelization configuration and a computing resource list for the cluster computing network, by one or more processing units, wherein the at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

In some embodiments, in the step (a), the partition indication data indicates the at least one biological information unit according to which of the sequencing data is capable of being partitioned into a plurality of consecutive, non-overlapping, variable-length segments so as to retain biological meaning of the sequencing data.

In some embodiments, the at least one biological information unit is at least one of chromosome, chromosome and discordant reads, centromere, or telomere.

In some embodiments, the at least one biological information unit includes a contiguous unmasked region.

In some embodiments, the at least one biological information unit includes a fixed length region.

In some embodiments, the at least one biological information unit includes protein coding genes.

In some embodiments, the at least one biological information unit includes genes.

In some embodiments, the at least one biological information unit includes a user-defined biological unit.

In some embodiments, in the step (b), each of the at least one recommendation list includes a plurality of computing resource entries, and a number of the computing resource entries of each of the at least one recommendation list is less than a number of computing resource entries included in the computing resource list.

In some embodiments, the partition indication data indicates the at least one biological information unit according to which of the sequencing data is capable of being partitioned into a plurality of consecutive, non-overlapping, variable-length segments so as to retain biological meaning of the sequencing data.

In some embodiments, the at least one recommendation list comprises a recommendation list for at least one portion of the sequencing data analysis, the recommendation list includes a plurality of computing resource entries indicating estimated processing times and corresponding estimated costs with respect to the at least one portion of the sequencing data analysis.

In some embodiments, the at least one recommendation list comprises a plurality of recommendation lists for a plurality of portions of the sequencing data analysis, each of the recommendation lists includes a plurality of corresponding computing resource entries indicating estimated processing times and corresponding estimated costs with respect to a corresponding one of the plurality of portions of the sequencing data analysis.

In some embodiments, the cluster computing network is an on-premises cluster computing network or a cloud computing network.

The present disclosure provides a non-transitory storage medium having instructions therein, when executed, causing at least one processing unit to perform a method for facilitating optimization of a cluster computing network for sequencing data analysis using adaptive data parallelization, as exemplified in any one of the embodiments.

The present disclosure provides a system for facilitating optimization of a cluster computing network for sequencing data analysis using adaptive data parallelization, the system comprises a memory; and at least one processing unit coupled to the memory to perform operations. The operations include the following. (a) A data parallelization configuration for a sequencing data analysis is determined, based on sequencing data and a pipeline selection, wherein the data parallelization configuration includes partition indication data indicating at least one biological information unit according to which of the sequencing data is to be partitioned. (b) At least one recommendation list for a sequencing data analysis is determined, based on the data parallelization configuration and a computing resource list for the cluster computing network, wherein the at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

In some embodiments, in the operation (a), the partition indication data indicates the at least one biological information unit according to which of the sequencing data is capable of being partitioned into a plurality of consecutive, non-overlapping, variable-length segments so as to retain biological meaning of the sequencing data.

In some embodiments, the at least one biological information unit is at least one of chromosome, chromosome and discordant reads, centromere, or telomere.

In some embodiments, the at least one biological information unit includes a contiguous unmasked region.

In some embodiments, the at least one biological information unit includes a fixed length region.

In some embodiments, the at least one biological information unit includes protein coding genes.

In some embodiments, the at least one biological information unit includes genes.

In some embodiments, the at least one biological information unit includes a user-defined biological unit.

In some embodiments, in the operation (b), each of the at least one recommendation list includes a plurality of computing resource entries, and a number of the computing resource entries of each of the recommendation list is less than a number of computing resource entries included in the computing resource list.

In some embodiments, the partition indication data indicates the at least one biological information unit according to which of the sequencing data is capable of being partitioned into a plurality of consecutive, non-overlapping, variable-length segments so as to retain biological meaning of the sequencing data.

In some embodiments, the at least one recommendation list comprises a recommendation list for at least one portion of the sequencing data analysis, the recommendation list includes a plurality of computing resource entries indicating estimated processing times and corresponding estimated costs with respect to the at least one portion of the sequencing data analysis.

In some embodiments, the at least one recommendation list comprises a plurality of recommendation lists for a plurality of portions of the sequencing data analysis, each of the recommendation lists includes a plurality of corresponding computing resource entries indicating estimated processing times and corresponding estimated costs with respect to a corresponding one of the plurality of portions of the sequencing data analysis.

The present invention provides a method for facilitating optimization of a cluster computing network for sequencing data analysis using adaptive data parallelization. The method comprises the following steps. The cluster computing network is informed to create a private computing environment in the cluster computing network for a user. The cluster computing network is instructed to deploy a software system for facilitating optimization for sequencing data analysis using adaptive data parallelization in the private computing environment for the user so that the private computing environment is capable of executing the software system to perform operations. The operations include the following. (a) A data parallelization configuration for a sequencing data analysis is determined, based on sequencing data and a pipeline selection, wherein the data parallelization configuration includes partition indication data indicating at least one biological information unit according to which of the sequencing data is to be partitioned. (b) At least one recommendation list is determined for the sequencing data analysis, based on the data parallelization configuration and a computing resource list for the cluster computing network, wherein the at least one recommendation list is for a computing device to produce at least one resource allocation selection from the at least one recommendation list so that the cluster computing network, in response to the at least one resource allocation selection, performs the sequencing data analysis on the sequencing data according to the at least one resource allocation selection and the data parallelization configuration.

A non-transitory storage medium having instructions therein, when executed, causing at least one processing unit to perform a method for facilitating optimization of a cluster computing network for sequencing data analysis using adaptive data parallelization, as exemplified.

A system for facilitating optimization of a cluster computing network for sequencing data analysis using adaptive data parallelization. The system comprises a memory; and at least one processing unit coupled to the memory to perform operations. The operations include the following. The cluster computing network is informed to create a private computing environment in the cluster computing network for a user. The cluster computing network is instructed to install a software system for facilitating optimization for sequencing data analysis using adaptive data parallelization in the private computing environment for the user so that the private computing environment is capable of executing the software system to perform operations including: (a) determining a data parallelization configuration for a sequencing analysis, based on sequencing data and a pipeline selection, wherein the data parallelization configuration includes partition indication data indicating at least one biological information unit according to which of the sequencing data is to be partitioned; and (b) determining at least one recommendation list for the sequencing analysis, based on the data parallelization configuration and a computing resource list for the cluster computing network. The at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

The present disclosure provides methods and systems using an Adaptive Data Parallelization (ADP) strategy for sequence data analysis. Such methods and systems are applicable for de novo genome sequence assembly or resequencing (in part or whole). The execution time of sequence data analysis can be improved via Adaptive Data Parallelization (ADP) strategy.

Accordingly, one aspect of the present disclosure relates to a method for sequence data analysis, in which of the method comprises one or more data parallelization processes, and each data parallelization process comprises the steps of: (a) dividing, in a cluster computing network, sequence data into a plurality of data subsets, (b) distributing, in the cluster computing network, the plurality of data subsets to multiple computing nodes, and (c) processing, in the cluster computing network, the plurality of data subsets in parallel on the multiple computing nodes.

As described herein, the cluster computing network is a cloud-based computing or an on-premises cluster computing.

In some embodiment, the method described herein comprises one data parallelization process. Such method may be applicable for de novo genome sequence assembly or for genome resequencing (in part or whole). In some examples, the sequence data described in step (a) are in the form of sequence data generated from a sequence device. In some examples, the sequence data in step (a) are in the format of FASTQ files.

In some embodiments, the method described herein comprises two or more data parallelization processes. Such method is applicable for genome resequencing (in part or whole). The method may further comprise the steps of read mapping and variant calling, and optionally, annotation. The sequence data are in the form of sequence data generated from a sequence device or sequence data analysis, partially processed or processed data, and/or data files compatible with particular software programs.

In some embodiments, the sequence data in step (a) are in the format of FASTQ, BAM (Binary Alignment File), and/or VCF (Variant Call Format) files.

In some embodiments, the sequence data in step (a) are the sequence data (reads) files generated from a sequence device. The sequence data in step (a) may be in the format of FASTQ files.

In some embodiments, the sequence data in step (a) are the sequence data generated from read mapping. The sequence data may be in the format of BAM files. Read mapping may be performed using open source and/or proprietary software tools.

In some embodiments, the sequence data in step (a) are the sequence data generated from variant calling. The sequence data may be in the format of VCF files. Variant calling may be performed using open source and/or proprietary software tools.

Another aspect of the present disclosure relates to a method for resequencing. The method includes the steps of: (a) receiving, in a cluster computing network, sequence data (reads) generated by a sequence device, (b) dividing, in the cluster computing network, the sequence data into a first plurality of data subsets, (c) distributing, in the cluster computing network, the first plurality of data subsets to multiple computing nodes, (d) performing, in the cluster computing network, read mapping in parallel on the multiple computing nodes, and (e) performing, in the cluster computing network, variant calling in parallel on the multiple computing nodes, wherein the step (d) of performing read mapping comprises the steps of: (i) mapping the reads to a reference genome, (ii) sorting the mapped reads, (iii) dividing the mapped reads into consecutive, non-overlapping, variable-length segments by a user's choice, and (iv) distributing a second plurality of data subsets containing the consecutive, non-overlapping, variable-length segments to multiple computing nodes.

In some embodiments, the method described herein further comprises a step (f) of merging, after variant calling, the data subsets into one data file.

In some embodiments, the step (e) in the method described further comprises the steps of: (1) dividing, in the cluster computing network, the sequence data from variant calling into a third plurality of data subsets, (2) distributing, in the cluster computing network, the third plurality of data subsets to multiple computing nodes, and (3) performing, in the cluster computing network, annotation in parallel on multiple computing nodes. In some embodiments, the method further comprises a step (4) of merging, after annotation, the data subsets into one data file.

The multiple computing nodes described in the method are configured to work together in a cluster computing network. The cluster computing may be a cloud-based computing or an on-premises cluster computing.

In some embodiments, the first plurality of data subsets is saved to a respective plurality of individual FASTQ files. In some embodiments, the second plurality of data subsets is saved to a respective plurality of individual BAM files corresponding to that respective segment. In some embodiments, the third plurality of data subsets is saved to a respective plurality of individual VCF files.

In some embodiments, the number of segments described in step (ii) is determined by the number of respective computing cores (processors) in the cluster computing network.

In some embodiments, the number of segments described in step (ii) is determined by the size of the reference genome.

In some embodiments, the mapped reads described herein are divided into consecutive, non-overlapping, variable-length segments based on a region of interest in the genome.

In some embodiments, the mapped reads described herein are divided into consecutive, non-overlapping, variable-length segments by chromosomes in the genome. In a human genome, there are 22 autosomal chromosomes, 2 sex chromosomes, and/or 1 mitochondria DNA, and the number of partitions can be 24 (excluding mitochondria DNA) or 25 (including mitochondria DNA).

In some embodiments, the mapped reads described herein are divided into consecutive, non-overlapping, variable-length segments by the tandem repeats on chromosomes (centromeres and telomeres) in the genome. In a human genome, there are 48 centromeres/telomeres.

In some embodiments, the mapped reads described herein are divided into consecutive, non-overlapping, variable-length segments by contiguous unmasked regions in the genome. In the human genome reference hg19, there are about 79 contiguous unmasked regions (greater than 100,000 bps).

In some embodiments, the mapped reads described herein are divided into consecutive, non-overlapping, variable-length segments by inter-chromosomes in the genome.

In some embodiments, the mapped reads in the method described herein are divided into consecutive, non-overlapping, variable-length segments by a combination of chromosomes, centromeres, telomeres, contiguous unmasked regions, and/or inter-chromosomes in the genome.

Advantageously, the method described herein is more likely to overcome the concern of having a loss of biologically significant information.

Another aspect of the present disclosure relates to a flexible and extensive workflow for resequencing. The workflow comprises the steps of: (a) deploying a software container into a cluster computing network, (b) receiving, in the cluster computing network, sequence data (reads) generated by a sequence device, (c) dividing, in the cluster computing network, the sequence data into a first plurality of data subsets, (d) performing read mapping, in the cluster computing network, in parallel on the multiple computing nodes using one or more software programs in the software container by user's choice, (e) performing variant calling, in the cluster computing network, in parallel on the multiple computing nodes using one or more software programs in the software container by user's choice, and (f) optionally, performing annotation, in the cluster computing network, in parallel on the multiple computing nodes using one or more software programs in the software container by user's choice, in which of the step (d) of read mapping comprises the steps of: (i) mapping the reads to a reference genome, (ii) sorting the mapped reads, (iii) dividing the mapped reads into consecutive, non-overlapping, variable-length segments by user's choice, and (iv) distributing a second plurality of data subsets containing the consecutive, non-overlapping, variable-length segments to multiple computing nodes.

In some embodiments, each of the multiple computing nodes in the workflow described herein has a common set of software applications installed thereon.

In some embodiments, the step (e) of performing variant calling in the workflow described herein uses the sorted list of aligned reads.

In some embodiments, each of the multiple computing nodes in the workflow described herein is coupled to the cluster computing network.

In some embodiments, the mapped reads in the workflow described herein are divided into consecutive, non-overlapping, variable-length segments based on a region of interest in the genome.

In some embodiments, each of the multiple computing nodes in the workflow described herein has a common set of software applications installed thereon.

In some embodiments, each of the multiple computing nodes in the workflow described herein is coupled to the cluster computing network.

In some embodiments, the number of consecutive, non-overlapping, variable-length segments in the workflow described herein is determined by the number of respective computing cores (processors) in the cluster computing network.

In some embodiments, the number of consecutive, non-overlapping, variable-length segments in the workflow described herein is determined by the size of the reference genome.

In some embodiments, the mapped reads in the workflow described herein are divided into consecutive, non-overlapping, variable-length segments based on a region of interest in the genome.

In some embodiments, the mapped reads in the workflow described herein are divided into consecutive, non-overlapping, variable-length segments by chromosomes in the genome.

In some embodiments, the mapped reads in the workflow described herein are divided into consecutive, non-overlapping, variable-length segments by centromeres and telomeres in the genome.

In some embodiments, the mapped reads in the workflow described herein are divided into consecutive, non-overlapping, variable-length segments by contiguous unmasked regions in the genome.

In some embodiments, the mapped reads in the workflow described herein are divided into consecutive, non-overlapping, variable-length segments by inter-chromosomes in the genome.

In some embodiments, the mapped reads in the workflow described herein are divided into consecutive, non-overlapping, variable-length segments by a combination of chromosomes, centromeres, telomeres, contiguous unmasked regions, and/or inter-chromosomes in the genome.

In some embodiments, the genome in the workflow described herein is a human genome.

In some embodiments, the software programs in the workflow described herein comprises at least one read mapping software used for mapping reads to a large reference genome. In some embodiments, the read mapping software is Burrows-Wheeler aligner (BWA).

Another aspect of the present disclosure relates to a system for sequence data analysis. The system comprises (a) a cluster computing network, (b) a master computing unit for receiving sequencing data (reads) for a sequence device, (c) a plurality of computing nodes for parallel processing data in the cluster computing network, each node comprising a processor, and (d) a software container comprising software programs for sequence data analysis, in which each of the plurality of computing nodes has the same set of software programs installed thereon, and the multiple computing nodes are configured in the cluster computing network to execute the software programs.

In some embodiments, the software programs described herein comprises one or more software programs for read mapping.

In some embodiments, the software programs described herein comprises one or more software programs for variant calling.

In some embodiments, the software programs described herein comprises one or more software programs for annotation.

The performance of methods, workflows and systems of the disclosure may be improved with the aid of various optimizations. Both software optimizations and hardware optimizations may be utilized.

The details of one or more embodiments of the disclosure are set forth in the description below. Other features or advantages of the present disclosure will be apparent from the following drawings and detailed description of several embodiments, and also from the appended claims.

### BRIEF DESCRIPTION OF THE DRAWINGS

FIG. 1 (PRIOR ART) shows a block-diagram, dataflow representation of a conventional sequencing data analysis.

FIG. 2 (PRIOR ART) is a schematic diagram illustrating loss of biologically significant information in the process of fixed-length partitioning during a conventional sequencing data analysis.

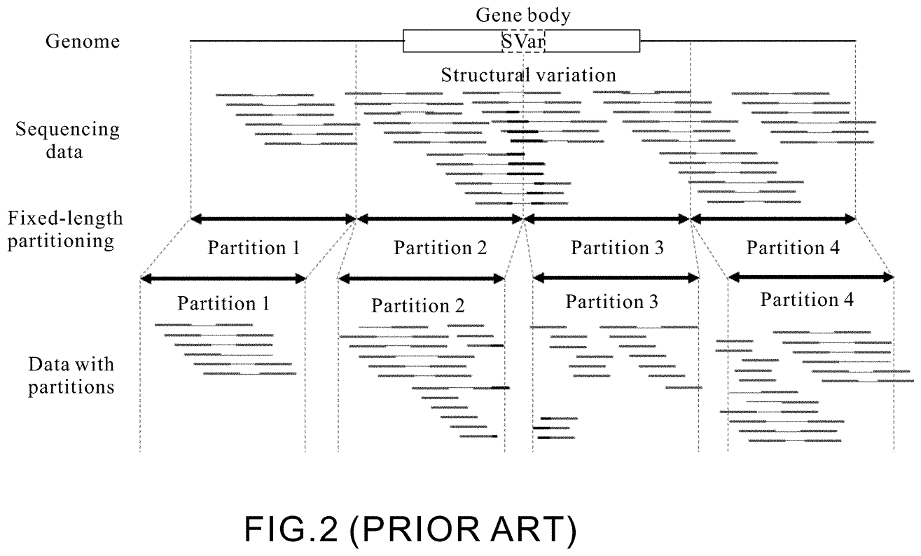

FIG. 3 is a block diagram illustrating a cluster computing network is to be utilized for performing sequencing data analysis, according to various embodiments.

FIG. 4A is a flowchart illustrating a method for facilitating optimization of a cluster computing network for sequencing data analysis using adaptive data parallelization according to an embodiment.

FIG. 4B is a flowchart illustrating a method for facilitating optimization of a cluster computing network for sequencing data analysis using adaptive data parallelization according to another embodiment.

FIG. 5A is a block diagram illustrating a cluster computing network to be utilized for performing sequencing data analysis, according to another embodiment.

FIG. 5B is a flowchart illustrating a method for facilitating optimization of a cluster computing network for sequencing data analysis using adaptive data parallelization according to an embodiment.

FIG. 6 is a block diagram illustrating a system for facilitating optimization of a cluster computing network for sequencing data analysis using adaptive data parallelization according to an embodiment.

FIG. 7 is a block-diagram, dataflow representation of an adaptive data parallelization method according to an embodiment of the present disclosure.

FIG. 8 is a schematic diagram illustrating a partition strategy for sequencing data according to an embodiment of the present disclosure.

FIG. 9 is a flowchart illustrating a process for identifying a data parallelization mechanism implemented by an adaptive data parallelization (ADP) module of FIG. 6 according to an embodiment.

FIG. 10 is a block diagram illustrating a pre-trained consumption model (PCM) determination module of FIG. 6 according to an embodiment.

FIG. 11 is a block diagram illustrating an adaptive resource recommendation (ARR) determination module of FIG. 6 according to an embodiment.

FIG. 12 is a schematic diagram illustrating a computing resource list according to an embodiment.

FIG. 13 is a schematic diagram illustrating a user interface indicating a recommendation list for variant calling according to an embodiment.

FIG. 14 is a schematic diagram illustrating an example of adaptive resource recommendation.

FIG. 15 is a schematic diagram illustrating elasticity of cluster computing that can be achieved by way of the method based of FIG. 4A, 4B, or 6.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed in practicing the invention.

The term “sequencing” generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA).

The term “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

The term “next generation sequencing” or “NGS” refers to sequencing technologies having increased throughput as compared to traditional Sanger and capillary electrophoresis-based approaches, for example with the ability to generate hundreds of thousands of relatively small sequencing reads at a time. Some examples of next generation sequencing techniques include, but are not limited to, sequencing by synthesis, sequencing by ligation, and sequencing by hybridization.

The term “genome” generally refers to an entirety of an organism's hereditary information. A genome can be encoded either in DNA or in RNA. A genome can comprise regions that code for proteins as well as non-coding regions. A genome can include the sequence of all chromosomes together in an organism. For example, the human genome has a total of 46 chromosomes. The sequence of all of these together constitutes the human genome.

The term “read” generally refers to a sequence of sufficient length (e.g., at least about 30 base pairs (bp)) that can be used to identify a larger sequence or region, e.g., that can be aligned to a location on a chromosome or genomic region or gene.

The term “coverage” generally refers to the average number of reads representing a given nucleotide in a reconstructed sequence. It can be calculated from the length of the original genome (G), the number of reads (N), and the average read length (L) as N*L/G. For instance, sequence coverage of 30× means that each base in the sequence has been read 30 times.

The term “alignment” generally refers to the arrangement of sequencing reads to reconstruct a longer region of the genome. Reads can be used to reconstruct chromosomal regions, whole chromosomes, or the whole genome.

The terms “variant” or “polymorphism” and generally refers to one of two or more divergent forms of a chromosomal locus that differ in nucleotide sequence or have variable numbers of repeated nucleotide units. Each divergent sequence is termed an allele, and can be part of a gene or located within an intergenic or non-genic sequence. The most common allelic form in a selected population can be referred to as the wild-type or reference form. Examples of variants include, but are not limited to single nucleotide polymorphisms (SNPs) including tandem SNPs, small-scale multi-base deletions or insertions, also referred to as indels or deletion insertion polymorphisms or DIPs), Multi-Nucleotide Polymorphisms (MNPs), Short Tandem Repeats (STRs), deletions, including microdeletions, insertions, including microinsertions, structural variations, including duplications, inversions, translocations, multiplications, complex multi-site variants, copy number variations (CNV). Genomic sequences can comprise combinations of variants. For example, genomic sequences can encompass the combination of one or more SNPs and one or more CNVs.

The term “calling” generally refers to identification. For example, “base calling” means identification of bases in a polynucleotide sequence, “SNP calling” generally means the identification of SNPs in a polynucleotide sequence, “variant calling” means the identification of variants in a genomic sequence.

The term “raw genetic sequence data” or “sequence data from sequence device” generally refers to unaligned genetic sequencing data, such as from a genetic sequencing device. In an example, raw genetic sequence data following alignment yields genetic information that can be characteristic of the whole or a coherent portion of genetic information of a subject for which of the raw genetic sequence data was generated. Genetic sequence data can include a sequence of nucleotides, such as adenine (A), guanine (G), thymine (T), cytosine (C) and/or uracil (U). Genetic sequence data can include one or more nucleic acid sequences. In some cases, genetic sequence data includes a plurality of nucleic acid sequences, at least some of which can overlap. For example, a first nucleic acid sequence can be (5′ to 3′) AATGGGC and a second nucleic acid sequence can be (5′ to 3′) GGCTTGT. Genetic sequence data can have various lengths and nucleic acid compositions, such as from one nucleic acid in length to at least 5, 10, 20, 30, 40, 50, 100, 1000, 10,000, 100,000, or 1,000,000 base pairs (double or single stranded) in length.

Methods, workflows and systems provided herein can be used with genetic data, such as deoxyribonucleic acid (DNA) or ribonucleic acid (RNA) data. Such genetic data can be provided by a sequence device, such as, with limitation, an Illumina, Pacific Biosciences, Oxford Nanopore, or Life Technologies (Ion Torrent) sequence device. Such devices may provide a plurality of raw genetic data corresponding to the genetic information of a subject (e.g., human), as generated by the device from a sample provided by the subject. In some situations, systems and methods provided herein may be used with proteomic information. Since there are over three billion base pairs (sites) on a human genome, sequencing a whole genome generates more than 100 gigabytes of data in BAM (the binary version of sequence alignment/map) and VCF (Variant Call Format) file formats.

The term “parallel computing” refers to the simultaneous use of multiple computing resources to solve a computational problem.

The term “cloud computing” generally refers to computing that occurs in environments with dynamically scalable and often virtualized resources, which typically include networks that remotely provide services to client devices that interact with the remote services. For example, cloud computing environments often employ the concept of virtualization as a preferred paradigm for hosting workloads on any appropriate hardware. The cloud computing model has become increasingly viable for many enterprises for various reasons, including that the cloud infrastructure may permit information technology resources to be treated as utilities that can be automatically provisioned on demand, while also limiting the cost of services to actual resource consumption. Moreover, consumers of resources provided in cloud computing environments can leverage technologies that might otherwise be unavailable. Thus, as cloud computing and cloud storage become more pervasive, many enterprises will find that moving data centers to cloud providers can yield economies of scale, among other advantages.

The term “cluster computing network” refers to a network connecting multiple stand-alone computers (nodes) to make large parallel computing.

While the methods, workflows and systems described herein constitute exemplary embodiments of the current disclosure, it is to be understood that the scope of the claims are not intended to be limited to the disclosed forms, and that changes may be made without departing from the scope of the claims as understood by those of ordinary skill in the art. Further, while objects and advantages of the current embodiments have been discussed, it is not necessary that any or all such objects or advantages be achieved to fall within the scope of the claims.

Whole Genome Sequencing

Whole genome sequencing such as next generation sequencing (NGS) enables faster, more accurate characterization of any species compared to traditional methods, such as Sanger sequencing. NGS data analysis involves in multiple computational steps, including primary analysis and secondary analysis to go from raw sequencing instrument output to variant discovery.

Primary analysis typically encompasses the process by which instrument-specific sequencing measures are converted into files containing the raw genetic sequence data (short reads), including generation of sequencing run quality control metrics. These instrument specific primary analysis procedures have been well developed by the various NGS manufacturers and can occur in real-time as the raw data is generated. With the HiSeq instrument, primary analysis for whole human genome comparative sequencing (resequencing) produces about one billion raw genetic sequence data (short reads).

Secondary analysis relates to data analysis for raw genetic sequence data generated from the primary sequence. Typically, there are two ways of secondary analysis:

(1) De novo sequencing: De novo sequencing refers to sequencing a novel genome where there is no reference sequence available for alignment. In the case of wild animals and new pathogens, because no reference sequences exist for these genomes, whole-genome sequencing must be newly performed in each case.

(2) Resequencing: Resequencing is when an organism's genome is sequenced and assembly is done using the reference genome as a template. For example, with humans this would be the genome produced by the Human Genome Project. The key reason for carrying out resequencing is to compare differences between genomes from the same species. Genomes consisting of high-precision reference sequences have been prepared for humans and mice. In the age of next-generation sequencing (NGS), by using these genomes, the genome sequence and the sequence of an exon region (exome) of a certain individual can be determined and reference genome sequences mapped using the homogeny of sequences as an index. For humans, diseases may be diagnosed and treated based on information about conformational polymorphisms (individual genome information) that can be obtained through comparison with the corresponding reference genome sequence.

Resequencing typically encompasses computational steps including: (1) Read Mapping: alignment of the raw genetic sequence data (short reads) to a reference genome, and (2) Variant Calling: variant calling from that alignment to detect differences between the patient sample and the reference. This process of detection of genetic differences, variant detection and genotyping, enables the scientific and clinical communities to accurately use the sequence data to identify single nucleotide polymorphisms (SNPs), small insertions and deletion (indels) and structural changes in the DNA, such as copy number variants (CNVs) and chromosomal rearrangements, and optionally (3) Annotation.

A variety of software tools have been developed for read mapping, the alignment of the sequencing reads to a reference genome (i.e. aligners), and for variant calling from that alignment (i.e. variants callers).

BWT-based (Bowtie, BWA) and hash-based (MAQ, Novoalign, Eland) aligners (mapper) have been most successful so far. Among them BWA is a popular choice due to its accuracy, speed, the ability to take FASTQ (a text-based format for storing both a biological sequence and its corresponding quality scores) input and output data in Sequence Alignment/Map (SAM) format or a BAM format (a BAM file is a compressed SAM file), and the open source nature.

Picard and SAMtools are typically utilized for the post-alignment processing steps and to output SAM binary (BAM) format files (See, Li, H. et al. The Sequence Alignment/Map format and SAMtools. Bioinformatics 25, 2078-2079 (2009), the disclosure of which is incorporated herein by reference).

Several statistical methods have been developed for genotype calling in NGS studies (see, Nielsen, R., Paul, J. S., Albrechtsen, A. & Song, Y. S. Genotype and SNP calling from next-generation sequencing data. Nat Rev Genet 12, 443-451 (2011)), yet for the most part, the community standard for human genome resequencing is BWA alignment with the Genome Analysis Toolkit (GATK) for variant calling (Depristo, 2011). Among the many publicly available variant callers, GATK has been used in the 1000 Genome Project. It uses sophisticated statistics in its data processing flow: local realignment, base quality score recalibration, genotyping, and variant quality score recalibration. The results are variant lists with recalibrated quality scores, corresponding to different categories with different false discovery rates (FDR).

The majority of studies utilizing next generation sequencing to identify variants in human diseases have utilized this combination of alignment with BWA, post alignment processing with SAMtools and variant calling with GATK (See, Gonzaga-Jauregui, C., Lupski, J. R. & Gibbs, R. A. Human genome sequencing in health and disease Annu Rev Med 63, 35-61 (2012), the disclosure of which is incorporated herein by reference).

Cluster Computing System for Sequencing Data Analysis

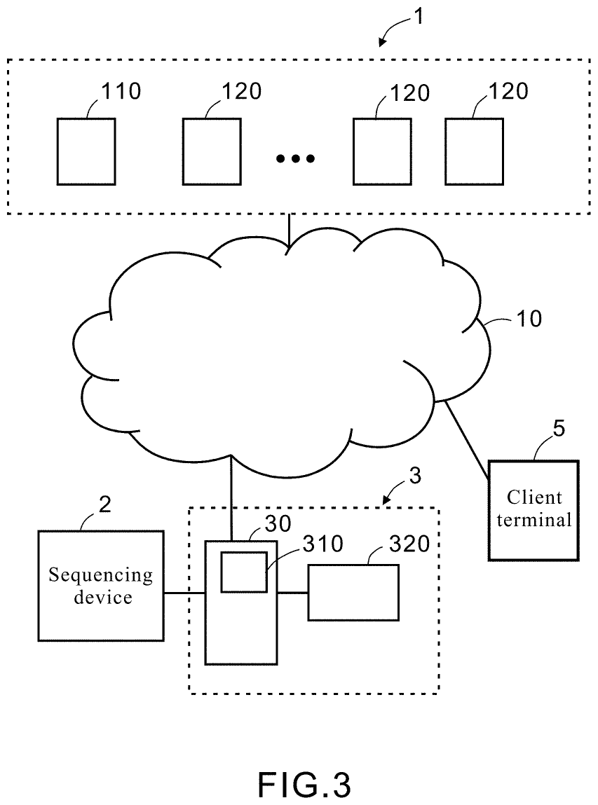

FIG. 3 is a block diagram illustrating a cluster computing system is to be utilized for performing sequencing data analysis, according to various embodiments. As shown in FIG. 3, a cluster computing system 1 is to be utilized for providing a parallel computing environment for performing sequencing data analysis, such as variant calling, or read mapping and variant calling, in a data parallelization approach. The cluster computing system 1 can be implemented by one or more cluster computing networks, such as an on-premises cluster, a cloud computing system (public or private), or a grid computing system, or a combination thereof (such as hybrid cloud computing platform, including an on-premises cluster and a cloud computing environment).

For any specific implementation of the cluster computing system 1 for performing sequencing data analysis in a data parallelization approach, computing resource allocation is a common issue related to efficiency and cost-effectiveness for the sequencing data analysis. The cluster computing system 1 provides shared computing resources, such as data storage (or cloud storage) and computing power. Specifically, an allocation of the shared computing resources for a user or a specified task or set of tasks can be indicated by computing component parameters, for example, including the number of available computing units (or CPU, core, virtual CPU or virtual core (vCPU or vCore)), memory capacity (e.g., capacity of primary memory (such as RAM) for program access), storage capacity (e.g., capacity of secondary memory (such as hard disk, flash disk, and so on), etc. Examples of computing resource allocations can be: 16 vCPUs, 64 GB RAM, 400 GB storage; 16 CPUs, 112 GB RAM, 224 GB storage; 32 CPUs, 128 GB RAM, 256 GB storage.

In a cloud computing environment, for example, a sequencing data analysis on specified sequential data, typically in tens or hundreds of gigabytes of data, can be done with different time and cost when a different computing resource scheme is allocated. A cloud computing platform provider generally offers various computing resource allocation plans, which are associated with respective prices, or provides various pricing plans, which are directly or indirectly corresponding to respective computing resource allocations. It is inevitably required to make a selection, either interactively with the user or automatically by software configuration or determination, from at least one computing resource list, which may include computing resource entries (e.g., tens or hundreds of entries such as 10, 20, 30, 50, 100 or more), each entry including a combination of computing component parameters, such as the number of computing units (or CPU, cores, vCore), an amount of memory capacity, an amount of storage capacity, etc., for a user to choose for performing their computing tasks. An appropriate computing resource for performing a sequencing data analysis is critical because sequencing data is typically in tens or hundreds of gigabytes of data and different computing resource allocations will affect the time and the cost for obtaining the results of the sequencing data analysis significantly.

In another example, in an on-premises cluster, although the total CPU number and machine type of the on-premises cluster may be fixed, the same issue of computing resource allocation is concerned. When a user of the on-premises cluster is going to process their NGS data, the user does not know how to assign the computing resource for performing sequencing data analysis. In a situation, the user A may assign almost all computing resource (even higher priority) for tasks of sequencing data analysis due to the expectation of efficiency. Although the user A′ tasks can be performed smoothly, the other user's tasks will be affected or even not to be able to be executed due to the occupation of the computing resource by the user A's tasks.

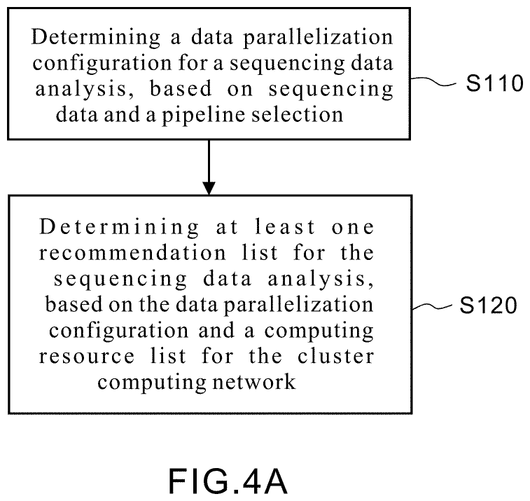

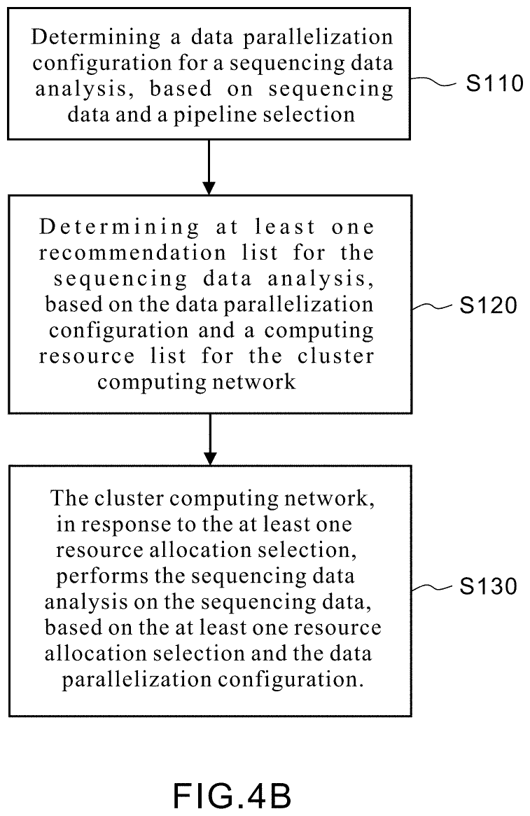

As such, the technology according to the present disclosure, as will be exemplified later by way of FIG. 4A, 4B, or other embodiments, facilitates computing resource allocation optimization of a cluster computing network for sequencing data analysis using adaptive data parallelization. The sequencing data analysis can be performed by using an optimized computing resource allocation and an adaptive data parallelization approach, without biological meaning loss.

FIG. 4A is a flowchart illustrating a method for facilitating optimization of a cluster computing network for sequencing data analysis using adaptive data parallelization according to an embodiment. When a sequencing data analysis is to be performed on sequencing data by a cluster computing network, the method can be executed to adaptively obtain a data parallelization configuration and at least one recommendation list, automatically. The cluster computing network can be configured to perform the sequencing data analysis, in a data parallelization approach according to the data parallelization configuration and in a resource allocation according to at least one entry from at least one recommendation list. The method comprises the following steps.

As shown in step S110, a data parallelization configuration for a sequencing data analysis is determined, based on sequencing data and a pipeline selection, by one or more processing units. The data parallelization configuration includes partition indication data indicating at least one biological information unit, according to which of the sequencing data is to be partitioned. For example, sequencing data is a whole genome.

As shown in step S120, at least one recommendation list for the sequencing data analysis is determined, based on the data parallelization configuration and a computing resource list for the cluster computing network, by one or more processing units. The at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

In step S110, the method as illustrated in FIG. 4A facilitates that the sequencing data analysis can be performed by using a computing resource allocation and an adaptive data parallelization approach, without biological meaning loss. As a result, the sequencing data analysis can be achieved with efficiency and cost-effectiveness and without biological meaning loss.

In some embodiments, in the step S110, the partition indication data indicates the at least one biological information unit according to which of the sequencing data is capable of being partitioned into a plurality of consecutive, non-overlapping, variable-length segments so as to retain biological meaning of the sequencing data.

In some embodiments, the at least one biological information unit is at least one of chromosome, chromosome and discordant reads, centromere, or telomere.

In some embodiments, the at least one biological information unit includes a contiguous unmasked region. For example, in a human genome, there exists a plurality of regions whose functions are unknown, which can be referred to as contiguous “masked region” in the context. Conversely, a region in the human genome between any two consecutive “masked regions” can be called a contiguous unmasked region. When the at least one biological information unit indicates a plurality of contiguous unmasked regions, the sequencing data can be partitioned at the contiguous masked regions. In this way, the biological meaning loss can be reduced or avoided.

In some embodiments, the at least one biological information unit includes a fixed length region. For example, the fixed length region indicates a data amount equal to 1 MB or above. Certainly, the implementation of the invention is not limited to the examples.

In some embodiments, the at least one biological information unit includes protein coding genes.

In some embodiments, the at least one biological information unit includes genes.

In some embodiments, the at least one biological information unit includes a user-defined biological unit.

In some embodiments, in the step S120, each of the at least one recommendation list includes a plurality of computing resource entries, and a number of the computing resource entries of each of the at least one recommendation list is less than a number of computing resource entries included in the computing resource list.

In some embodiments, the partition indication data indicates the at least one biological information unit according to which of the sequencing data is capable of being partitioned into a plurality of consecutive, non-overlapping, variable-length segments so as to retain biological meaning of the sequencing data. For example, in the step S120, the at least one recommendation list is determined based on the number of the plurality of consecutive, non-overlapping, variable-length segments according to the data parallelization configuration and the computing resource entries included in the computing resource list.

In some embodiments, step S120 can be implemented to determine the at least one recommendation list comprising a recommendation list for a preprocess stage (e.g., read mapping) of the sequencing data analysis, the recommendation list includes a plurality of computing resource entries indicating estimated processing times and corresponding estimated costs (e.g., 2.4 hours and USD 50; 1.6 hours and USD 48; 4 hours and USD 42) with respect to the preprocess stage of the sequencing data analysis.

In some embodiments, step S120 can be implemented to determine the at least one recommendation list comprising a recommendation list for an analysis stage (e.g., variant calling) of the sequencing data analysis, the recommendation list includes a plurality of computing resource entries indicating estimated processing times and corresponding estimated costs (e.g., 1.2 hours and USD 25; 0.82 hours and USD 32; 2.02 hours and USD 22) with respect to the analysis stage of the sequencing data analysis.

Certainly, the implementation of step S120 is not limited to the examples. In some embodiments, step S120 can be implemented to determine a plurality of recommendation lists for a plurality of portions of the sequencing data analysis. Each of the recommendation lists includes a plurality of corresponding computing resource entries indicating estimated processing times and corresponding estimated costs with respect to a corresponding one of the plurality of portions of the sequencing data analysis. For example, the sequencing data analysis can divided into a plurality of portions (or stages), or a plurality of portions (or stages) of the sequencing data analysis are required or allowed to be performed adaptively according to respective resource allocations. For example, a sequencing data analysis can be regarded as having a plurality of stages such as: read mapping stage and variant calling stage; read mapping stage, variant calling stage, and annotation stage; read mapping stage and annotation stage; or variant calling stage and annotation stage. Each portion (or stage) of the sequencing data analysis is associated with at least a corresponding one of the plurality of recommendations lists. Each of the corresponding recommendation list(s) with respect to that portion (or stage) of the sequencing data analysis includes a plurality of computing resource entries indicating estimated processing times and corresponding estimated costs (e.g., 1.2 hours and USD 25; 0.82 hours and USD 32; 2.02 hours and USD 22). For different portion (or stage) of the sequencing data analysis, a corresponding resource allocation selection can be produced, either interactively with the user or automatically by software configuration or determination, from the corresponding recommendation list(s) with respect to that portion (or stage) of the sequencing data analysis. In this manner, the sequencing data analysis can be performed adaptively according to various resource allocation selections for different portions (or stage) of the sequencing data analysis, in contrast to performing the sequencing data analysis according to a fixed resource allocation. As a result, the sequencing data analysis can be achieved with efficiency and cost-effectiveness in an adaptive manner.

In some embodiments, the cluster computing network is an on-premises cluster computing network or a cloud computing network.

In some embodiments, a system for facilitating optimization of a cluster computing network for sequencing data analysis using adaptive data parallelization is provided. The system comprises a memory; and at least one processing unit coupled to the memory to perform a plurality of operations including operations corresponding to steps S110 and S120, exemplified in one of the embodiments based on FIG. 4A in the present disclosure or any combination thereof, whenever appropriate.

In some embodiments, a system for facilitating optimization of a cluster computing network for sequencing data analysis using adaptive data parallelization can be configured in various forms. Referring to FIG. 3, the cluster computing system can be utilized for performing sequencing data analysis in various practical applications or scenarios, according to various embodiments. In an embodiment, the cluster computing system 1 can be utilized for providing a parallel computing environment for performing sequencing data analysis obtained from a sequencing device. For example, a sequencing device 2 and an analytic computing unit 3 are presented in FIG. 3. For a given sample, the sequencing device 2 outputs a plurality of sequence “reads”, sequence data, in terms of a list of bases. The analytic computing unit 3 is configured to receive and perform data processing on the sequence data for further sequencing analysis by way of bioinformatics techniques, for example, by executing one or more application programs using one or more processing units 310 of a computing unit 30; the analysis output can be further presented on a display device 320 visually by graphical interfaces or schematic diagrams, or statistically by charts or bars, or in terms of indications of the bases in string form. In addition, the analytic computing unit 3 can communicate with the cluster computing system 1 via a communication network 10 (e.g., a local area network, the Internet, or any appropriate wired or wireless network, or a combination thereof) in order to perform sequencing data analysis more efficiently by using a plurality of computing units (such as computing units (110, 120)) in the cluster computing system 1, such as a cloud computing environment or an on-premises cluster or other cluster computing environment. In an example, before the sequencing data analysis is performed, the method based on FIG. 4A can be executed to facilitate computing resource allocation optimization of the cluster computing system 1 for sequencing data analysis using adaptive data parallelization. In the example, at least one recommendation list is determined by the method based on FIG. 4A and the analytic computing unit 3 can be served as the “computing device” to produce at least one resource allocation selection from the at least one recommendation list, as specified in step S120. In this manner, the sequencing data analysis can be performed by using an optimized computing resource allocation and an adaptive data parallelization approach, without biological meaning loss.

For example, the sequencing device 2, such as a Next Generation Sequencer (NGS), a third generation DNA sequencer, a nucleic acid sequencer, a polymerase chain reaction (PCR) machine, or a protein sequencing device, is used to automate the DNA or RNA or protein (DNA/RNA/protein) sequencing process. For example, the sequencing device 2 can be configured to sequence a plurality of nucleic acid fragments obtained from a single biological sample and generate a data file containing a plurality of fragment sequence reads that are representative of the genomic profile of the biological sample.

In another embodiment, a client terminal 5 can be linked to the cluster computing system 1 to request for sequencing data analysis by uploading sequencing data files. The client terminal 5 can be a thin client or thick client computing device. In various embodiments, client terminal 5 can execute a web browser (e.g., CHROME, INTERNET EXPLORER, FIREFOX, SAFARI, etc.) or an application program that can be used to request the cluster computing system 1 for the analytic operations. In some examples, before the sequencing data analysis is performed, the client terminal 5 can be configured to execute the method based on FIG. 4A and communicate with the cluster computing system 1 or the cluster computing system 1 (e.g., computing unit 110 or 120) can be configured to execute the method based on FIG. 4A and communicate with the client terminal 5, so as to configure operating parameters (e.g., data parallelization selection, computing resource allocation, etc.) for sequencing data analysis, depending on the requirements of a particular application or implementation of the cluster computing system 1. In the examples, at least one recommendation list is determined by the method based on FIG. 4A and the client terminal 5 can be served as the “computing device” to produce at least one resource allocation selection from the at least one recommendation list, as specified in step S120. The client terminal 5 can also display results of the sequencing data analysis after the sequencing data analysis is performed.

In various embodiments, the analytics computing unit 3 or client terminal 5 can be a computing device, such as a server, a workstation, a personal computer, a mobile device, etc. The cluster computing system 1 is implemented by a plurality of computing devices. For example, the computing device includes one or more computing units (such as CPU, graphical processing unit (GPU), tensor processing unit (TPU)), a memory, and a communication unit (e.g., wired or wireless network module for communicating with other computing device).

FIG. 4B is a flowchart illustrating a method for facilitating optimization of a cluster computing network for sequencing data analysis using adaptive data parallelization according to another embodiment. In this embodiment, the method of FIG. 4B, based on FIG. 4A, further includes step S130 in which of the cluster computing network (such as the cluster computing system 1), in response to the at least one resource allocation selection, performs the sequencing data analysis on the sequencing data, according to the at least one resource allocation selection and the data parallelization configuration.

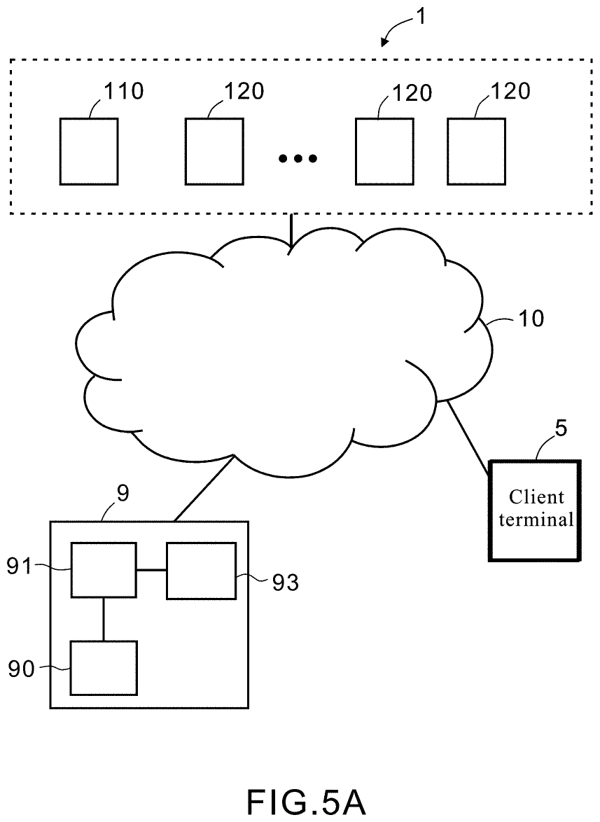

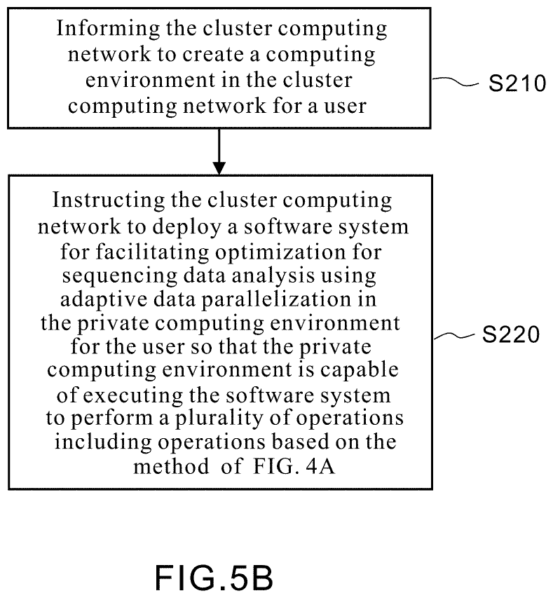

FIG. 5A is a block diagram illustrating a cluster computing network that is to be utilized for performing sequencing data analysis, according to another embodiment. In FIG. 5A, a system 9 for facilitating optimization of a cluster computing network for sequencing data analysis using adaptive data parallelization is provided. The system 9 comprises a memory 90; and at least one processing unit 91 coupled to the memory 90 to perform a plurality of operations including operations as illustrated in a method of FIG. 5B. In addition, the system 9 may further comprise a communication unit 93 for communicating with the communication network 10 or the cluster computing system 1, in a wired or wireless manner.

Referring to FIG. 5B, a method for facilitating optimization of a cluster computing network for sequencing data analysis using adaptive data parallelization according to an embodiment is illustrated.

As shown in step S210, the system 9 informs the cluster computing network (such as the cluster computing system 1) to create a computing environment (such as a private computing environment) in the cluster computing network for a user.

As shown in step S220, the system 9 instructs the cluster computing network (such as the cluster computing system 1) to deploy a software system for facilitating optimization for sequencing data analysis using adaptive data parallelization in the private computing environment for the user so that the private computing environment is capable of executing the software system to perform a plurality of operations including operations based on the method of FIG. 4A.

The following provides various embodiments based on the method of FIG. 4A.

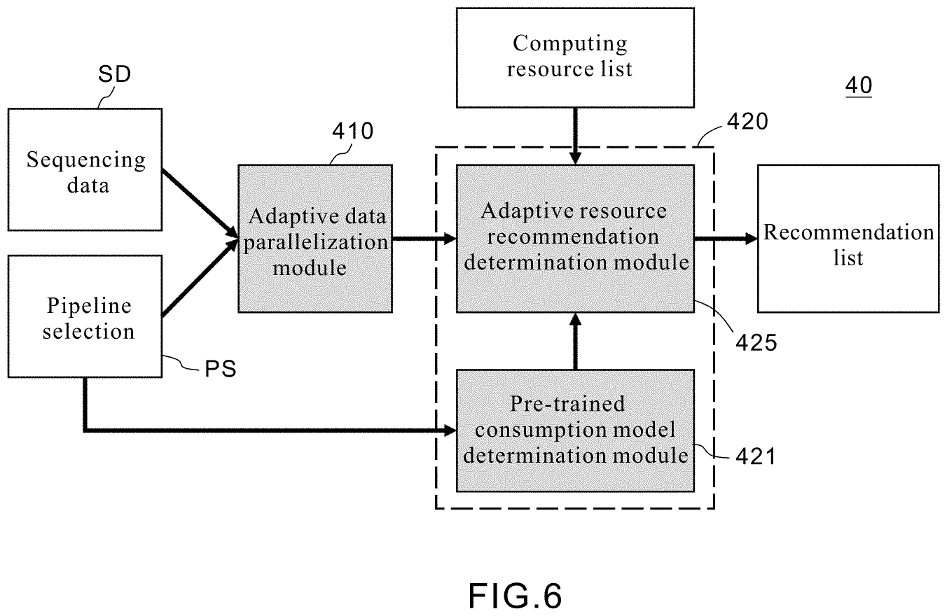

FIG. 6 is a block diagram illustrating a system 40 for facilitating optimization of a cluster computing network for sequencing data analysis using adaptive data parallelization according to an embodiment. The system 40 is an implementation of the method based on FIG. 4A, and can be implemented by way of software modules or processes, or so on, which are executable by one or more computing units.

In FIG. 6, the system 40 includes an adaptive data parallelization (ADP) module 410 and an adaptive resource recommendation (ARR) module 420. The adaptive resource recommendation (ARR) module 420 includes a pre-trained consumption model (PCM) determination module 421 and an adaptive resource recommendation (ARR) determination module 425. Before sequencing data (SD) is processed, the ADP module 410 is configured to implement step S110 based on the method of FIG. 4A so as to determine a data parallelization configuration (such as a most suitable one for the sequencing data) based on both data volume of the sequencing data SD and a pipeline selection (PS), wherein the pipeline selection is selected by a user through a user profile, a default value, or an interactive selection in a software interface, for example. The data parallelization configuration affects a data parallelization mechanism, in which of the huge amount of the sequencing data is able to be split into tens to hundreds of small data chunks (or partitions) without loss of any biological meanings. In addition, the PCM determination module 421 pre-trains computation consumption and resource requirement of the pipeline selection, resulting in a pre-trained consumption model (PCM), which can be represented by a data structure including a plurality of parameters, and can be utilized in the ARR module 420. The ARR module 420 is configured to implement step S120 based on the method of FIG. 4A. Therefore, the ARR module 420 will generate at least one recommendation list (such as several objective-oriented plans) based on the sequencing data, the data parallelization configuration, the pre-trained consumption model, and a computing resource list for the cluster computing network, wherein the cluster computing network, such as infrastructure as a service (IaaS) provider (e.g. Amazon AWS, Google Cloud, Microsoft Azure, etc.), provides the computing resource list indicating accessible computing resource entries.

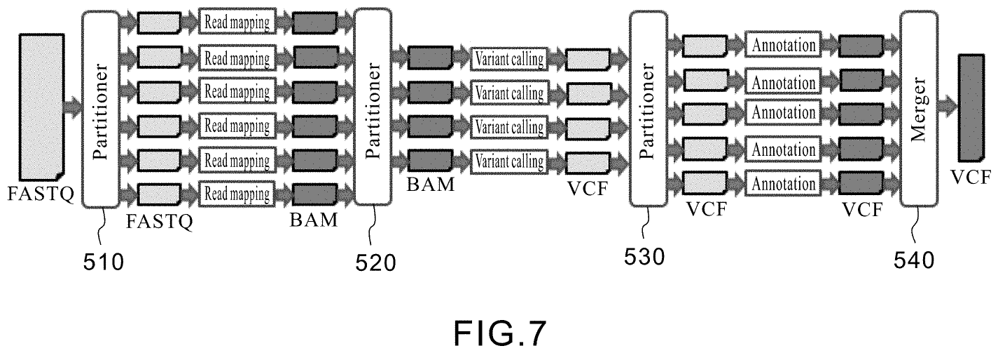

In order to demonstrate how the data parallelization configuration affects a data parallelization mechanism that will be utilized in the sequencing data analysis the following description is provided. Referring to FIG. 7, a block-diagram, dataflow representation of an adaptive data parallelization method is illustrated according to an embodiment of the present disclosure.

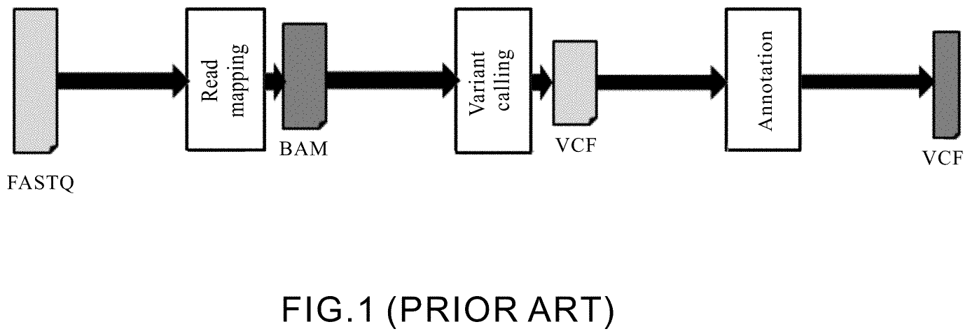

For example, the sequencing data of NGS is usually recorded in a single file and two paired files for Single-End and Paired-End sequencing, respectively. Take a paired-end 30× WGS sample for example, all of the sequencing data will be stored into two files by FASTQ format. Each of them has more than 500M reads. The conventional approach of the sequencing data processing is non-data-parallelization model, as shown in FIG. 1. It means that each data processing stage (such as read mapping, variant calling, and annotation) will take all of the data into a single process. Although some bioinformatic tools are able to support multi-threading, most of them are incapable of being executed in a parallel manner in distributed clusters.

As shown in FIG. 7, using a data parallelization model without modifying the existing bioinformatic tools can speed up the process of the sequencing data analysis of NGS data. The following provides several examples with respect to a preprocess stage and an analysis stage.

For example, in a preprocessing stage, such as a read mapping stage, the huge file in FASTQ format, for example, is split gently and properly into tens to hundreds of small data chunks. A given partitioner 510 must make sure the data partitioning process is performed without loss of any biological meanings. Therefore, all of the small data chunks are able to be processed for read mapping in parallel within a single computing unit by multi-threading or across multiple computing nodes (such as the computing units 110, 112) in a parallel computing manner, so as to obtain a plurality of files in BAM format.

For example, after the read mapping stage, in an analysis stage, such as a variant calling stage, the files in BAM format, for example, are partitioned by a partitioner 530 into a plurality of segments in files in BAM format so as to retain biological meaning of the sequencing data. The partitioner 530 performs partitioning according to the at least one biological information unit indicated by the partition indication data as specified in step S120 of the method based on FIG. 4A so as to ensure the data partitioning process is performed without loss of any biological meanings. In this manner, all of the segments are able to be processed for variant calling in parallel within a single computing unit by multi-threading or across multiple computing nodes (such as the computing units 110, 112) in a parallel computing manner, resulting in a plurality of files in VCF format.

For example, after the variant calling stage of the analysis stage, the files in VCF format, for example, can be further partitioned optionally by a partitioner 540 into a plurality of files in VCF format so as to perform annotation, resulting in a plurality of files in VCF format. The files in VCF format after annotation can then be merged by a merger 540, resulting in a file in VCF format, for example.

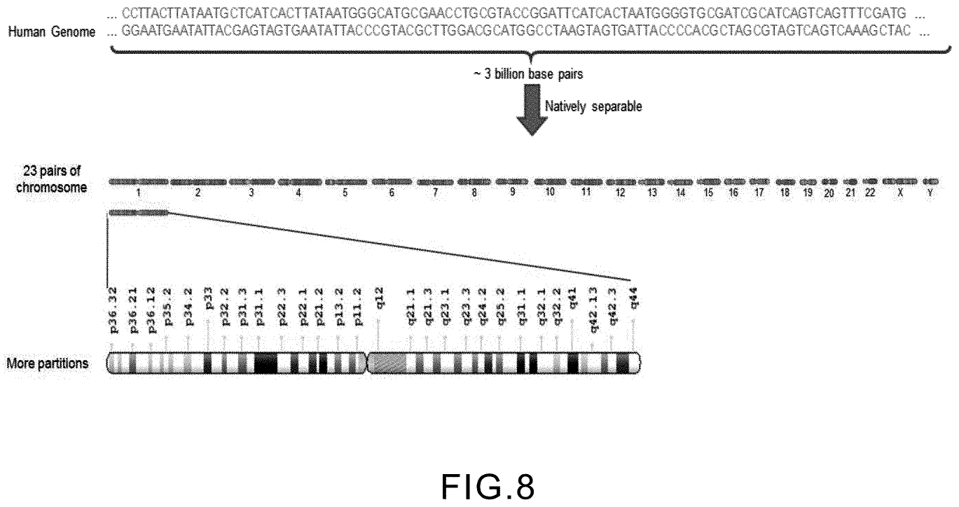

FIG. 8 is a schematic diagram illustrating a partition strategy for sequencing data according to an embodiment of the present disclosure. As illustrated above with respect to FIG. 7, in the analysis stage, partitioning is performed according to the at least one biological information unit indicated by the partition indication data as specified in step S120 of the method based on FIG. 4A so as to ensure the data partitioning process without loss of any biological meanings. In an embodiment, the at least one biological information unit can be taken so that the sequencing data can be partitioned into a plurality of consecutive, non-overlapping, variable-length segments so as to retain biological meaning of the sequencing data.

Take human genome for example, there are 23 pairs of chromosomes (22 pairs of autosomes and one pair of sex chromosomes). The at least one biological information unit can be taken as 23 pairs of chromosomes. Therefore, all of the alignment records (such as the files after read mapping) are able to be separated into 23 partitions without loss of any biological meanings. Furthermore, the data are able to be partitioned by 25,000 genes if protein coding genes are only considered.

TABLE 1 lists a plurality of partitioning methods based on different kinds of biological information units. For example, when Chromosomes are taken as the biological information units, the number of partitions is 24, the average length of each partition is about 128,000,000, and the speed of sequencing data analysis for variant calling will be 10 times faster than the reference of only 1 partition.

TABLE 1
Adaptive data parallelization strategies

| | | Average | | |
|---|---|---|---|---|
| | Number | Length | | |
| Partitioning | of | of each | Maximal | |
| Method | Partitions | partition | Length | Speedup |
| Single Collapsed | 1 | 3,079,843,747 | 3,079,843,747 | 1X |
| Partition | | | | |
| Chromosome | 24 | ~128,000,000 | 247,199,719 | >10X |
| Chromosome | 25 | ~128,000,000 | 247,199,719 | >10X |
| Discordant Reads | | | | |
| Centromere/ | 48 | ~64,000,000 | ~125,000,000 | >20X |
| telomere | | | | |
| Contiguous | 79 | ~39,000,000 | ~80,000,000 | >40X |
| Unmasked | | | | |
| Regions | | | | |
| (>100,000 bps) | | | | |
| 1M Fixed Length | 3101 | 1,000,000 | 1,000,000 | >1,000X |
| Regions | | | | |
| Protein Coding | ~21,000 | ~10-15K | 2,220,381 | >1,000X |
| Genes | | | | |
| Genes | ~50,000 | ~10-15K | 2,220,381 | >1,000X |

In some embodiments of the invention, the data parallelization method can be adaptively according to the given data analysis pipeline selection. There are several predefined data parallelization methods (e.g., partitioning methods as illustrated in TABLE 1) based on HG19. Taken a human Reference Genome for example, GRCh38 has 77 non-overlapping and non-padding genome regions; each region does not contain over continuous 10,000 Ns. In some embodiments, the length of each partition can be at least more than read length.

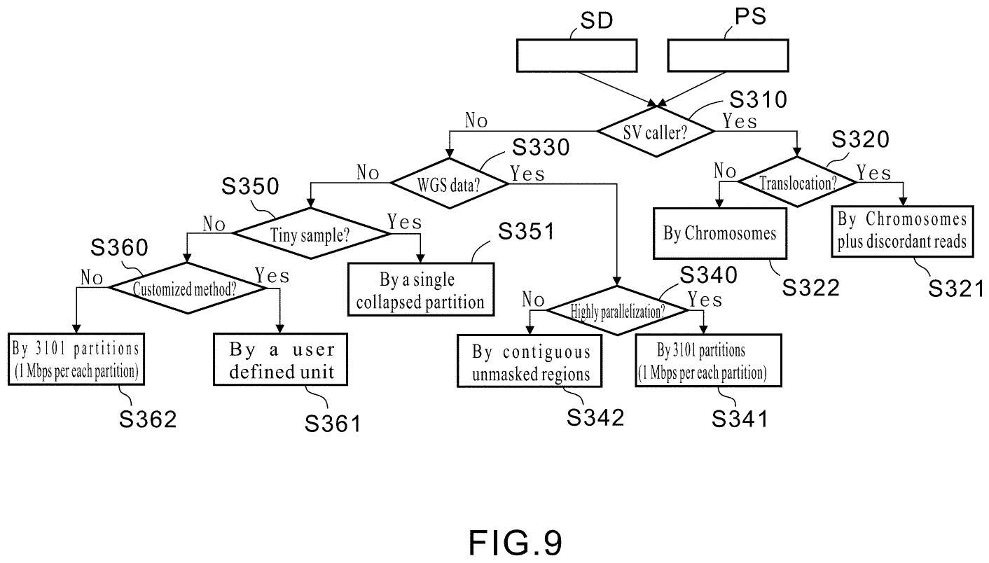

FIG. 9 is a flowchart illustrating a process for identifying (or determining) a data parallelization mechanism implemented by an adaptive data parallelization (ADP) module of FIG. 6 according to an embodiment. The process is an embodiment of step S10 of FIG. 4A. According to the volume of sequencing data and the pipeline selection (which indicates the chosen pipeline), the ADP module 410 can be configured to generate a data parallelization configuration indicating the most suitable data parallelization method, according to the process of FIG. 9. For example, the pipeline selection can be generated by default setting, by a user profile, or by using a software interface providing selections about pipelining for the user to choose, and so on. The pipeline selection can be implemented as a data structure (such as an array, a matrix, a profile, or data in any appropriate form) to indicate information for pipelining in the sequencing data analysis, such as: whether read mapping and variant calling pipelines are selected (or indicated by the file type of the sequencing data: FASTQ), or variant calling pipeline is needed (or indicated by the file type of the sequencing data: BAM), and so on; one or more pipelines, corresponding to specific algorithm(s) for sequencing data analysis, used in the sequencing data analysis for variant detection; and whether the tool(s) is parallelization friendly. The data parallelization configuration can be implemented by a data structure (such as an array, a matrix, a profile, or data in any appropriate form) to indicate information for performing data parallelization of read mapping (e.g., FASTQ chunking) and/or variant calling (e.g., BAM partitioning), for example, partition indication data indicating at least one biological information unit according to which of the sequencing data is to be partitioned, corresponding to the partitioning method as illustrated in TABLE 1.

Referring to FIG. 9, firstly, as shown in step S310, it is determined whether the pipeline selection indicates that a caller (i.e. a bioinformatic software tool) to be used in the sequencing data analysis is for structural variant calling or not. If so, the process proceeds to step S320 in which it is determined whether translocation is considered. If not, the process goes to step S330. In step S320, if translocation is considered, the data parallelization configuration is taken by Chromosomes plus discordant reads, as shown in step S321. If translocation is not considered, the data parallelization configuration is taken by Chromosomes, as shown in step S322.

In step S310, if it is determined that the caller is not a caller for structural variation, it means that the caller is for SNP/Indel calling, the data type or data volume will be the next criterion. The data volume can be categorized into a plurality of tiers, for examples, whole genome sequencing (WGS), whole exome sequencing (WES), and targeted panel, which are respectively in the size ranges of hundreds of GB, tens of GB, smaller than 10 GB. As shown in step S330, it is checked whether the data volume or size of the sequencing data is for WGS data. If it is for WGS data, step S340 is performed in which a determination is made whether a highly parallelization pipeline, which corresponds to at least a bioinformatic tool, is selected. Some bioinformatic tools are known to be highly parallelization by design, e.g. Google Deepvariant and GATK4 GenotypeGVCFs. In an example, variant-callers are categorized into a highly parallelization type and a normal type; once a highly parallelization pipeline is selected, the data parallelization configuration is taken by 3101 partitions (1 Mbps per each partition), for example, in step S341. In this way, the highly parallelization method can be applied to reduce the execution time significantly when computing resources are sufficient. If the highly parallelization is not selected, the data parallelization configuration is taken by contiguous unmasked regions, in step S342.

In step S330, if the pipeline selection is not for WGS data, step S350 is performed to check whether the sequencing data is a tiny sample. If the sequencing data is a tiny sample (e.g., the sequencing data is a tiny sample if corresponding FASTQ file size smaller than 5 GB), there is no need to perform data partitioning because each data partition method brings a certain amount of computational overhead, wherein the data parallelization configuration is taken by a single collapsed partition, in step S351. If the sequencing data is not a tiny sample, step S360 is performed to check whether a customized method is selected. If the customized method is selected, the data parallelization configuration is taken by a user defined unit, in step S361, so as to increase the flexibility of ADP. If the customized method is not selected, the data parallelization configuration is taken by 3101 partitions (1 Mbps per each partition), in step S362.

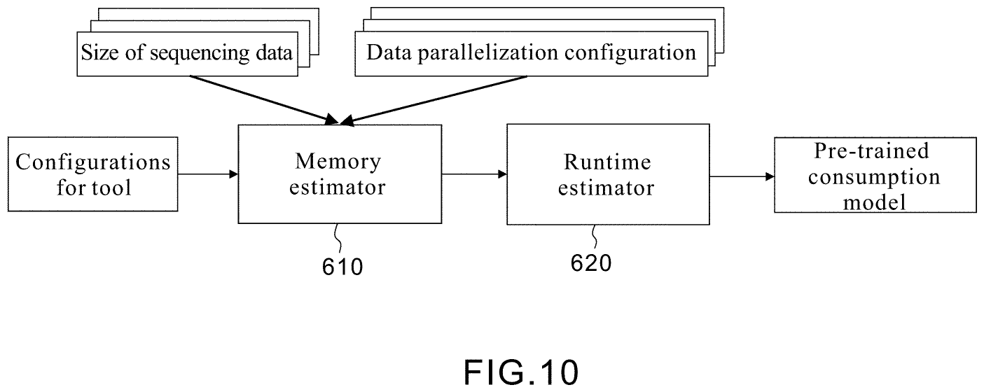

FIG. 10 is a block diagram illustrating a pre-trained consumption model (PCM) determination module of FIG. 6 according to an embodiment. PCM determination module determines a PCM, which can be represented by a data structure including a plurality of parameters, and will be utilized in the ARR module 420. For example, the PCM indicates how much time is required for a unit task with respect to resource requirement such as a memory amount and an amount of CPU or vCores.

As shown in FIG. 10, the PCM determination module includes a memory estimator 610 and a runtime estimator 620. The memory estimator 610 is used to evaluate the bioinformatic tools adopted in the chosen pipeline one-by-one based on chunked data (e.g., a piece of simulated sequencing data (i.e., a reference example for estimation), or size of input data (sequencing data), etc.) and all of suitable parallelization methods (e.g., partitioning methods as illustrated in TABLE 1).

In an example, the memory estimator 610 estimates the memory configuration of BWA MEM aligner, which is an alignment software tool for Burrows-Wheeler-Alignment using maximal exact matches algorithm, according to a threading configuration of the tool, as shown in Table 2. Table 2 illustrates an example of a memory estimation matrix for BWA MEM aligner corresponding to different threading configuration. As illustrated in Table 2, the amount of memory is estimated to increase as the number of threads to be used rises. Since BWA MEM aligner supports multithreading, if this aligner is executed in each of multiple computing units (e.g., as a virtual machine) of a cluster computing system, each of these computing units can be further performed alignment using multithreading in addition to cluster computing.

TABLE 2
Memory estimation matrix for BWA MEM aligner

| BWA MEM | Threads 1 | Threads 4 | Threads 16 |
|---|---|---|---|
| Memory | 7 GB | 7.2 GB | 7.4 GB |

In another example, the memory estimator 610 estimates the memory configuration of GATK4 GenotypeGVCFs cohort variant-caller according to the data parallelization configuration and a memory estimation matrix, as shown in Table 3. Table 3 illustrates an example of a memory estimation matrix for GATK4 GenotypeGVCFs cohort variant caller corresponding to different data partition configurations (e.g., as illustrated in Table 1). In Table 3, the numbers of partitions indicate how many partitions it is going to split the reference genome for different data partition configurations, wherein the more the partitions, the smaller the partition data amount. The memory estimator 610 accordingly provides a memory configuration according to the data parallelization configuration obtained from the ADP module 410. For example, when the data parallelization configuration indicates that a partition method of 3101 partitions is taken, the memory estimator 610 accordingly provides a memory configuration of 10 GB.

TABLE 3
Memory estimation matrix for GATK4 GenotypeGVCFs cohort
variant caller corresponding to different data partition configuration

| GATK4 | | | 3101 |
|---|---|---|---|
| GenotypeGVCFs | 25 partitions | 155 partitions | partitions |
| Memory | 30 GB | 20 GB | 10 GB |

Then, the runtime estimator 620 is used to generate the pre-trained consumption model for each tool based on the estimation of the memory estimator 610. The offline mode indicates that the PCM is pre-trained by a piece of simulated sequencing data, which is template data as a reference example for estimation. For example, the simulated sequencing data can be FASTQ data downloading from National Center for Biotechnology Information (NCBI), used to representing a sample FASTQ file for computation performance estimation.

In some embodiments, the PCM, which can be represented by a data structure including a plurality of parameters, and will be utilized in the ARR module 420. For example, the PCM indicates how much time is required for a unit task with respect to resource requirement such as a memory amount and an amount of CPU or vCores. In an example, the PCM trained off-line can be a matrix indicating the unit runtime for data chunks of different chunk size or different chromosomal regions, as shown in Table 4 and Table 5, and the memory configuration obtained by the memory estimator 610. Table 4 illustrates a runtime estimation matrix for BWA MEM aligner corresponding to different data chunk sizes on an Intel Skylake CPU. Table 5 illustrates a runtime estimation matrix for deepvariant variant-caller corresponding to different chromosomal partition size on an Intel Skylake CPU. Tables 4 and 5 can be obtained by experiment using a timer with respect to the simulated data as a reference basis, for example. In practical implementation, the data by Table 4 and 5 can be regarded as given or predetermined data.

TABLE 4
Runtime estimation matrix for BWA MEM aligner corresponding
to different data chunk sizes on Intel Skylake CPU.

| BWA MEM | 128 MB | 256 MB | 512 MB |
|---|---|---|---|
| Runtime | 3 minutes | 6 minutes | 12 minutes |

TABLE 5
Runtime estimation matrix for deepvariant variant-caller corresponding
to different chromosomal partition size on Intel Skylake CPU.

| deepvariant | 24 partitions | 155 partitions | 3101 partitions |
|---|---|---|---|
| Runtime | 2200 minutes | 267 minutes | 8 minutes |

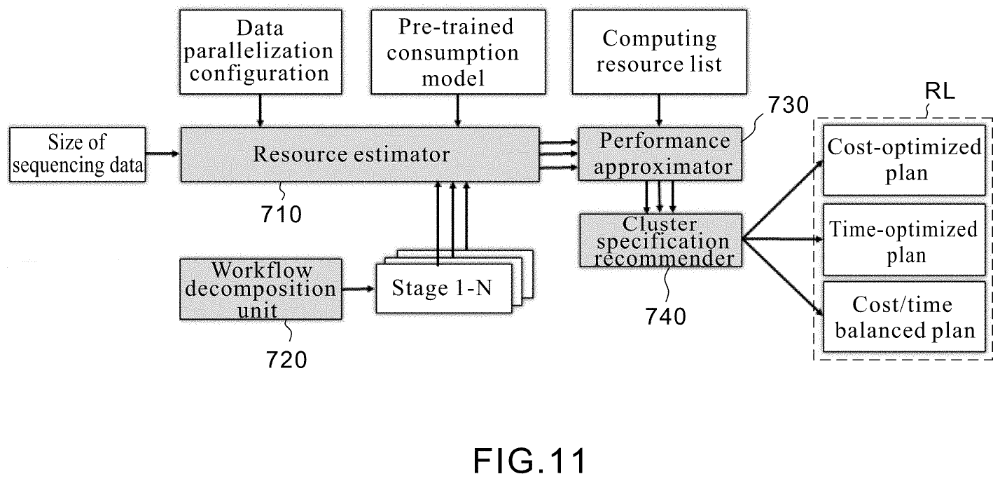

FIG. 11 is a block diagram illustrating an adaptive resource recommendation (ARR) determination module of FIG. 6 according to an embodiment.

As shown in FIG. 11, the ARR determination module includes a resource estimator 710, a workflow decomposition unit 720, a performance approximator 730, and a cluster specification recommender 740. First, the workflow decomposition unit 720 compiles the chosen pipeline into several processing stages. The key factor for pipeline decomposition is the data partitioning scheme, indicating by the pipeline selection or data parallelization configuration, for the input data (i.e. sequencing data). For implementation, the workflow decomposition unit 720 can be a determination as to whether a read-mapping stage and a variant-calling stage are required; or a variant-calling stage is required, for example. For example, the determination can be done by way of the file type of the sequencing data. For FASTQ files, indicating nucleotides sequences generated in parallel by NGS sequencer, the data are partitioned based on data chunk size. For BAM files, indicating reads aligned to different chromosomal regions, the data are partitioned based on the genome coordination. As such, in an example of GATK4 Germline short variant discovery (SNPs+Indels) pipeline, the workflow is decomposed by the workflow decomposition unit 720 into a FASTQ-to-BAM stage and a BAM-to-VCF stage to respectively achieve data parallelization for FASTQ and BAM files. If the sequencing data is a BAM file and variant calling is required for the sequencing data analysis only, the workflow decomposition unit 720 decomposes the workflow into a BAM-to-VCF stage. For implementation, the workflow decomposition unit 720 outputs data representing the workflow decomposition result (e.g., data indicating “stage 1” for a read mapping stage and “stage 2” for a variant calling stage; or “stage N” for any possible N-th stage (N>0)).

Then, the resource estimator 710 generates the computing consumption for each processing stage (such as read mapping, variant calling, or annotation) based on the volume or size of the sequencing data, the data parallelization configuration obtained by the ADP module 410, and the PCM suggested by the PCM determination module 421. Based on the pre-trained consumption model from the runtime estimator 620, a unit execution time of a partition can be estimated based on the configuration of the data chunk size or the genomic partition numbers, and the resource estimator 710 can estimate the total consumption by the product of the number of data partitions and the unit execution time of data partition. For example, for a FASTQ-to-BAM stage with 1,000 256 MB data chunks, the total needed CPU time will be 6,000 minutes.

By referring to the given computing resource list, the performance approximator 730 is able to calculate the computational consumption for each processing stage and also determine the cost and the execution time for each computing unit. For example, the computing resource list can be defined with VM type plus VM-number. In an example, the computing resource list indicates a predefined cluster configuration where the type of the virtual machines, whether it is GPU empowered, and the number of VM are listed, as shown in Table 6. The performance approximator 730 can estimate the execution times of the given workflow when the workflow is executed in clusters of different configurations.

TABLE 6
Computing resource list.

| Name of a cluster | | VM | Haying |
|---|---|---|---|
| configuration | VM type | number | GPU |
| 40d | Azure | 5 | No |
| | Standard_D13_V2 | | |
| 80d | Azure | 10 | No |
| | Standard_D13_V2 | | |
| 36g | Azure | 6 | YES |
| | Standard_NC6 | | |
| 72g | Azure | 12 | YES |
| | Standard_NC6 | | |

For example, when the FASTQ-to-BAM stage of 1,000 256 MB data chunks is executed on a 40d cluster, the 1,000 data chunks will be grouped into 25 batches, each of which will take 6 minutes of execution. As such, the approximated execution time for the FASTQ-to-BAM stage is 150 minutes in a 40d cluster. Same estimation can be applied for the rest items on the computing resource list to get the approximation for each combination of pipeline stages and cluster configurations.

Finally, the cluster specification recommender 740 will determine a recommendation list including three different cluster specifications based on three different objectives: cost-optimized, time-optimized and cost/time balanced.

Take the read mapping step for example, in some embodiments, the ARR module can be implemented based on the following equations.

For time optimization, the minimized time can be determined based on number of chunks (S) for input data, number of vCore (V) per computing unit, number of computing units (N) to be launched, and an average execution time (R) of the given pipeline per chunk. For time optimization, V and N can be determined under the equation (1):

$$Time = arg { min}_{V , N} [ \frac{S}{( V ⨯ N )} ] ⨯ R ,$$

and equation (2):

Cost=Time×N×C

For cost optimization, the minimized cost can be determined based on number of chunks (S) for input data, number of vCore (V) per computing unit, number of computing units (N) to be launched, an average execution time (R) of the given pipeline per chunk, and a cost (C) per hour for a computing unit. For time optimization, V and N can be determined under the equation (3):

$$Time = arg { min}_{V , N} [ \frac{S}{( V ⨯ N )} ] ⨯ R ⨯ N ⨯ C ,$$

and equation (4):

$$Time = \frac{Cost}{N ⨯ C} .$$

Take the variant calling step for example, in some embodiments, the ARR module can be implemented based on the following equations.

For time optimization, the minimized time can be determined based on the longest execution time (Rₘₐₓ) of the given pipeline by the given parallelization mechanism if number of partitions (P) in the given parallelization mechanism is less than or equal to number of vCore (V) per computing unit times number of computing units (N) to be launched. Otherwise, the minimized time can be determined based on the average execution time (Rₘₑₐₙ) of the given pipeline by the given parallelization mechanism, number of partitions (P) in the given parallelization mechanism, number of vCore (V) per computing unit, and number of computing units (N) to be launched. For time optimization, V and N can be determined under the following equations:

$$\begin{matrix}Time = arg { min}_{V , N} \{ \begin{matrix}{R}_{max} & if P \leq ( V ⨯ N ) ; \\ {R}_{mean} ⨯ ⌈ \frac{P}{( V ⨯ N )} ⌉ & otherwise\end{matrix} & ( 5 ) \\ Cost = Time ⨯ N ⨯ C & ( 6 )\end{matrix}$$

For cost optimization, V and N can be determined under the equations:

$$\begin{matrix}Cost = arg { min}_{V , N} \{ \begin{matrix}{R}_{max} ⨯ N ⨯ C & if P \leq ( V ⨯ N ) ; \\ {R}_{mean} ⨯ ⌈ \frac{P}{( V ⨯ N )} ⌉ ⨯ N ⨯ C & otherwise\end{matrix} & ( 7 ) \\ Time = \frac{Cost}{N ⨯ C} & ( 8 )\end{matrix}$$

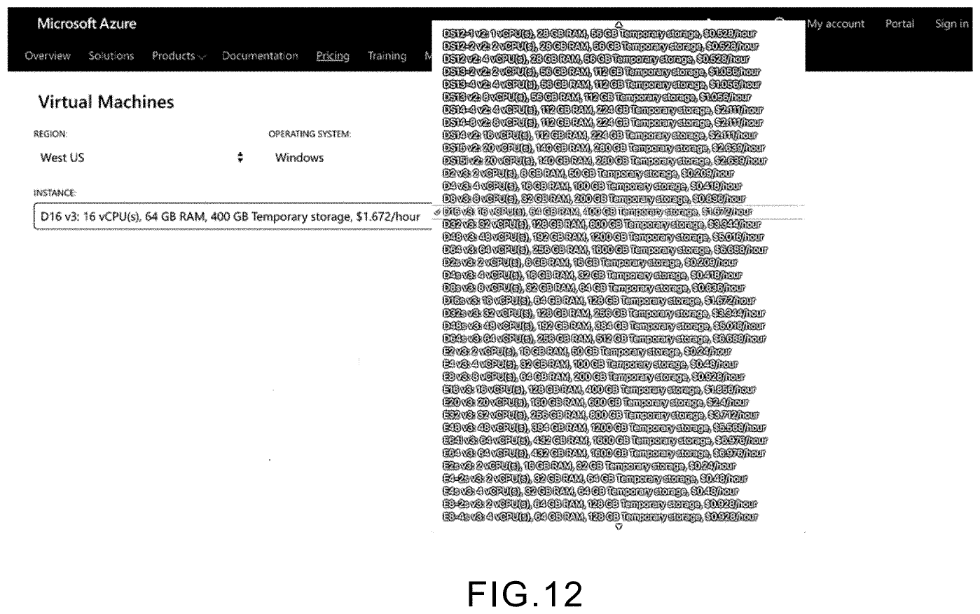

Table 6 is just an illustration of the computing resource list supporting two kinds of virtual machine types, and the computing resource list is not limited thereto. In other example, the computing resource list may include tens of computing units with different resource specification available on Microsoft Azure, as shown in FIG. 12.

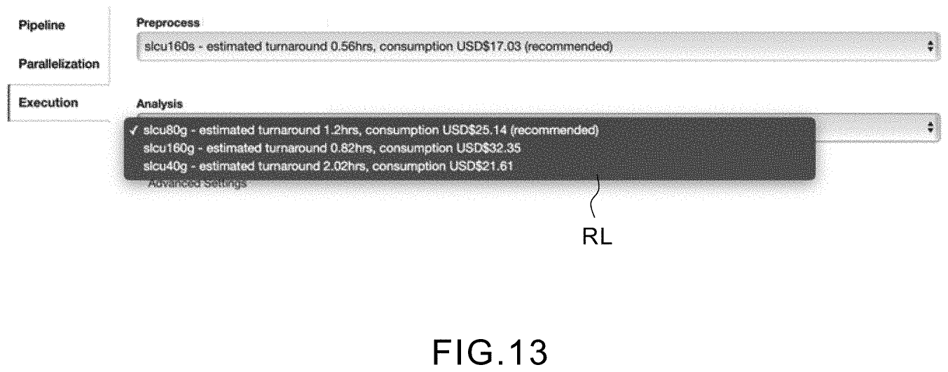

FIG. 13 is a schematic diagram illustrating a user interface indicating a recommendation list for variant calling according to an embodiment. As shown in FIG. 13, a recommendation list RL for an analysis stage (e.g., variant calling) is illustrated according to an embodiment. There are three cluster plans for variant calling step. S1cu80g means that a cluster with 80 vCores will be launched and the estimation of the execution time is 1.2 hours. In addition, the cost will be $25.14 USD. For Cost optimization, s1cu40 is suggested. For time optimization, s1cu160 is recommended. As can be compared, the computing resource list provided by the cloud computing provider includes entries each corresponding to number of cores, an amount of RAM, an amount of storage, and a rate of cost, while the recommendation list RL includes entries each corresponding to a cost and total time. In this way, the method based on FIG. 4A can be utilized to perform, before the sequencing data analysis is executed, to facilitate that the sequencing data analysis can be performed by using recommended computing resource and adaptive data parallelization, without biological meaning loss. As a result, the sequencing data analysis can be achieved with efficiency and cost-effectiveness and without biological meaning loss. In addition, by the method based on FIG. 4A, the computing resource list provided by the cloud computing provider is converted into a recommendation list in terms of different parameters so that a selection can be readily made interactively by the user. Alternatively, the selection can be made automatically by implementation of a software program for the selection based on a criterion when appropriate.

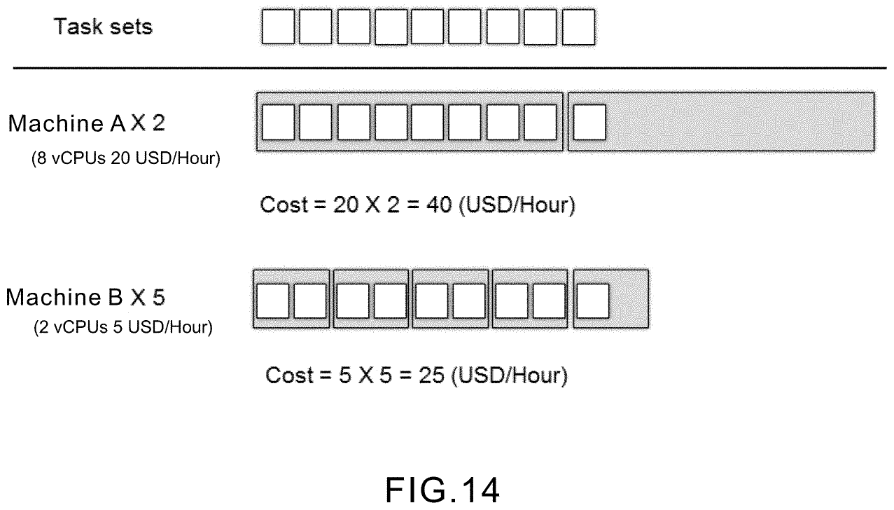

FIG. 14 is a schematic diagram illustrating an example of adaptive resource recommendation. In FIG. 14, the input data is split into 9 chunks, for example. In a current cloud provider providing a cluster computing network, two kinds of machine type are available, Machine A has 8 vCPUs and Machine B has only 2 CPUs. Therefore, the ARR module can propose to launch 2 Machine As or 5 Machine Bs. The execution time should be the same. However, the cost is quite different. Therefore, the ARR module will choose 5 Machine Bs for Cost-optimized cluster Specification.

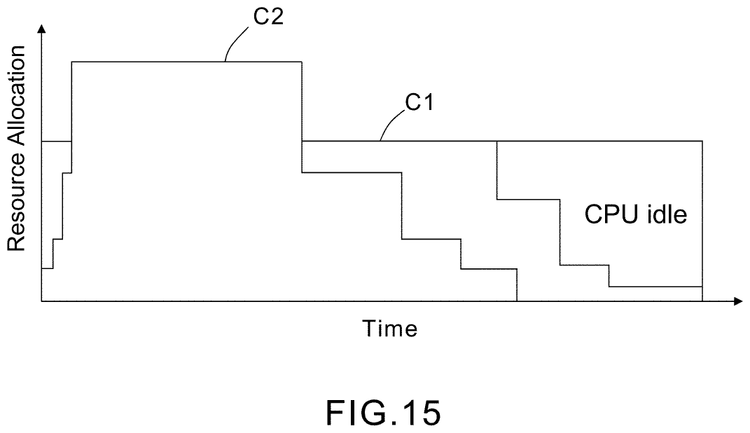

FIG. 15 is a schematic diagram illustrating elasticity of cluster computing that can be achieved by way of the method based on FIG. 4A, 4B, or 6. As shown in FIG. 15, in an implementation of a sequencing data analysis, the computing resource allocation is fixed, as represented by a curve C1, so that no support is provided for cohort analysis for multiple samples, only fixed data parallelization and fixed pipeline can be done, and it also results in an expensive cost. For example, in FIG. 15, when the CPU is idle, as illustrated in a right portion of the area below the curve C1, the computing resource being allocated is wasted. By contrast, in another implementation of the sequencing data analysis, the method based on FIG. 4A, 4B, or 6 is utilized and can facilitate adaptive computing resource allocation, as represented by a curve C2, so that the performance for the sequencing data analysis can be enhanced with less total time when the resource is sufficient and idle time for the computing resource can be adaptively reduced.

Adaptive Data Parallelization (ADP)

In order to accelerate the speed of sequence data analysis, the present disclosure provides methods, workflows and systems based on an innovative approach, Adaptive Data Parallelization (ADP), for rapid sequence data analysis. The methods, workflows and systems enable sequencing pipelines to be executed in parallel on a multi-node and/or multi-core compute infrastructure in a highly efficient manner.

Adaptive Data Parallelization (ADP) approach has an ability to change to suit different conditions for De Novo sequencing or resequencing or depending on a user's need.

For De Novo sequencing, after primary sequencing (e.g. initial DNA sequence), a partition process may be applied to divide reads into a plurality of sequencing pipelines, followed by De Novo assembly.

For resequencing, after primary sequencing (e.g. initial DNA sequence), a partition process may be applied to divide reads into a plurality of sequencing pipelines, preferably in FASTQ file format, followed by read mapping programs. After read mapping, a partition process may be applied to divide the sequence data into a plurality of sequencing pipelines, preferably in BAM file format, and followed by Variant Calling programs. After Variant Calling, a partition process may be applied to divide the input data into a plurality of sequencing pipelines preferably in VCF file format, and optionally followed by annotation programs.

Accordingly, the present disclosure relates to a method for sequence data analysis using adaptive data parallelization (ADP), in which of the method comprises one or more data parallelization processes, and each data parallelization process comprises the steps of: (a) dividing, in a cluster computing network, sequence data into a plurality of data subsets, (b) distributing, in the cluster computing network, the plurality of data subsets to multiple computing nodes, and (c) processing, in the cluster computing network, the plurality of data subsets in parallel on the multiple computing nodes.

As described herein, the cluster computing network is a cloud-based computing or an on-premises cluster computing.

In some embodiments, the method described herein comprises one data parallelization process. Such method may be applicable for de novo genome sequence assembly or for genome resequencing (in part or whole). In some examples, the sequence data described in step (a) are in the form of sequence data generated from a sequence device. In some examples, the sequence data in step (a) are in the format of FASTQ files.

In some embodiments, the method described herein comprises two or more data parallelization processes. Such method is applicable for genome resequencing (in part or whole). The method may further comprise the steps of read mapping and variant calling, and optionally, annotation. The sequence data are in the form of sequence data generated from a sequence device or sequence data analysis, partially processed or processed data, and/or data files compatible with particular software programs.

In some embodiments, the sequence data in step (a) are in the format of FASTQ, BAM (Binary Alignment File), and/or VCF (Variant Call Format) files.

In some embodiments, the sequence data in step (a) are the sequence data (reads) files generated from a sequence device. The sequence data in step (a) may be in the format of FASTQ files.

In some embodiments, the sequence data in step (a) are the sequence data generated from read mapping. The sequence data may be in the format of BAM files. Read mapping may be performed using open source and/or proprietary software tools.

In some embodiments, the sequence data in step (a) are the sequence data generated from variant calling. The sequence data may be in the format of VCF files. Variant calling may be performed using open source and/or proprietary software tools.

The use of such parallel processing sequence data can improve the performance of various analysis tasks in sequence analysis including, for example, identifying sequencing duplicates, identifying highest quality reads or read pairs in these duplicates, identifying motifs in sequences, determining read counts in specific genomic loci on a genome, and identifying allele variants and frequencies.

Methods For Resequencing

Another aspect of the present disclosure relates to a method for resequencing. The method includes the steps of: (a) receiving, in a cluster computing network, sequence data (reads) generated by a sequence device, (b) dividing, in the cluster computing network, the sequence data into a first plurality of data subsets, (c) distributing, in the cluster computing network, the first plurality of data subsets to multiple computing nodes, (d) performing, in the cluster computing network, read mapping in parallel on the multiple computing nodes, and (e) performing, in the cluster computing network, variant calling in parallel on the multiple computing nodes, wherein the step (d) of performing read mapping comprises the steps of: (i) mapping the reads to a reference genome, (ii) sorting the mapped reads, (iii) dividing the mapped reads into consecutive, non-overlapping, variable-length segments by a user's choice, and (iv) distributing a second plurality of data subsets containing the consecutive, non-overlapping, variable-length segments to multiple computing nodes.

In some embodiments, the method described herein further comprises a step (f) of merging, after variant calling, the data subsets into one data file.

In some embodiments, the step (e) in the method described further comprises the steps of: (1) dividing, in the cluster computing network, the sequence data from variant calling into a third plurality of data subsets, (2) distributing, in the cluster computing network, the third plurality of data subsets to multiple computing nodes, and (3) performing, in the cluster computing network, annotation in parallel on multiple computing nodes. In some embodiments, the method further comprises a step (4) of merging, after annotation, the data subsets into one data file.

The multiple computing nodes described in the method are configured to work together in a cluster computing network so that they can be viewed as a single system in a highly efficient manner. The cluster computing may be a cloud-based computing or an on-premises cluster computing.

In some embodiments, the first plurality of data subsets is saved to a respective plurality of individual FASTQ files. In some embodiments, the second plurality of data subsets is saved to a respective plurality of individual BAM files corresponding to that respective segment. In some embodiments, the third plurality of data subsets is saved to a respective plurality of individual VCF files.

In some embodiments, the number of segments described in step (iii) is determined by the number of respective computing cores (processors) in the cluster computing network.

In some embodiments, the number of segments described in step (iii) is determined by the size of the reference genome.

In some embodiments, the mapped reads described herein are divided into consecutive, non-overlapping, variable-length segments based on a region of interest in the genome.

In some embodiments, the mapped reads described herein are divided into consecutive, non-overlapping, variable-length segments by chromosomes in the genome. In a human genome, there are 22 autosomal chromosomes, 2 sex chromosomes, and/or 1 mitochondria DNA, and the number of partitions can be 24 (excluding mitochondria DNA) or 25 (including mitochondria DNA).

In some embodiments, the mapped reads described herein are divided into consecutive, non-overlapping, variable-length segments by the tandem repeats on chromosomes (centromeres and telomeres) in the genome. In a human genome, there are 48 centromeres/telomeres.

In some embodiments, the mapped reads described herein are divided into consecutive, non-overlapping, variable-length segments by contiguous unmasked regions in the genome. In the human genome reference hg19, there are about 79 contiguous unmasked regions (greater than 100,000 bps).

In some embodiments, the mapped reads described herein are divided into consecutive, non-overlapping, variable-length segments by inter-chromosomes in the genome.

In some embodiments, the mapped reads in the method described herein are divided into consecutive, non-overlapping, variable-length segments by a combination of chromosomes, centromeres, telomeres, contiguous unmasked regions, and/or inter-chromosomes in the genome.

Advantageously, the method described herein is more likely to overcome the concern of having a loss of biologically significant information.

The performance of the method of the disclosure may be improved with the aid of various optimizations. Both software optimizations and hardware optimizations may be utilized.

Flexible And Extensive Workflow For Resequencing

Another aspect of the present disclosure relates to a flexible and extensive workflow for resequencing. The workflow comprises the steps of: (a) deploying a software container into a cluster computing network, (b) receiving, in the cluster computing network, sequence data (reads) generated by a sequence device, (c) dividing, in the cluster computing network, the sequence data into a first plurality of data subsets, (d) performing read mapping, in the cluster computing network, in parallel on the multiple computing nodes using one or more software programs in the software container by the user's choice, (e) performing variant calling, in the cluster computing network, in parallel on the multiple computing nodes using one or more software programs in the software container by the user's choice, and (f) optionally, performing annotation, in the cluster computing network, in parallel on the multiple computing nodes using one or more software programs in the software container by the user's choice, in which of the step (d) of read mapping comprises the steps of: (i) mapping the reads to a reference genome, (ii) sorting the mapped reads, (iii) dividing the mapped reads into consecutive, non-overlapping, variable-length segments by the user's choice, and (iv) distributing a second plurality of data subsets containing the consecutive, non-overlapping, variable-length segments to multiple computing nodes.

In some embodiments, each of the multiple computing nodes in the workflow described herein has a common set of software applications installed thereon.

In some embodiments, the step (e) of performing variant calling in the workflow described herein uses the sorted list of aligned reads.

In some embodiments, each of the multiple computing nodes in the workflow described herein is coupled to the cluster computing network.

In some embodiments, the mapped reads in the workflow described herein are divided into consecutive, non-overlapping, variable-length segments based on a region of interest in the genome.

In some embodiments, each of the multiple computing nodes in the workflow described herein has a common set of software applications installed thereon.

In some embodiments, each of the multiple computing nodes in the workflow described herein is coupled to the cluster computing network.

In some embodiments, the number of consecutive, non-overlapping, variable-length segments in the workflow described herein is determined by the number of respective computing cores (processors) in the cluster computing network.

In some embodiments, the number of consecutive, non-overlapping, variable-length segments in the workflow described herein is determined by the size of the reference genome.

In some embodiments, the mapped reads in the workflow described herein are divided into consecutive, non-overlapping, variable-length segments based on a region of interest in the genome.

In some embodiments, the mapped reads in the workflow described herein are divided into consecutive, non-overlapping, variable-length segments by chromosomes in the genome.

In some embodiments, the mapped reads in the workflow described herein are divided into consecutive, non-overlapping, variable-length segments by centromeres and telomeres in the genome.

In some embodiments, the mapped reads in the workflow described herein are divided into consecutive, non-overlapping, variable-length segments by contiguous unmasked regions in the genome.

In some embodiments, the mapped reads in the workflow described herein are divided into consecutive, non-overlapping, variable-length segments by inter-chromosomes in the genome.

In some embodiments, the mapped reads in the workflow described herein are divided into consecutive, non-overlapping, variable-length segments by a combination of chromosomes, centromeres, telomeres, contiguous unmasked regions, and/or inter-chromosomes in the genome.

In some embodiments, the genome in the workflow described herein is a human genome.

In some embodiments, the software programs in the workflow described herein comprises at least one read mapping software used for mapping reads to a large reference genome. In some embodiments, the read mapping software is Burrows-Wheeler aligner (BWA).

In some embodiments, the parallel processing paths may correspond, at least in part to at least some of 22 autosomal chromosomes and 2 sex chromosomes. In a further detailed embodiment, the analyzing step may include at least 24 parallel processing paths, where each of the at least 24 parallel processing paths corresponding to a respective one of the plurality of 22 autosomal chromosomes and 2 sex chromosomes. Alternatively, or in addition, the parallel processing paths may further correspond to read pairs with both mates mapped to different chromosomes.

In another alternative embodiment of the aspect, the analyzing step may include at least one step divided into at least 24 parallel processing paths, where each of the at least 24 parallel processing paths respectively correspond to 22 autosomal chromosomes and 2 sex chromosomes.

In another alternative embodiment of this aspect, the analyzing step may involve a step of mapping reads to a reference genome, where the step of mapping reads to the reference genome may also be divided into a plurality of parallel processing paths.

In another alternative embodiment of this aspect, the method may include processing a plurality of subsets of the genetic sequence data among the plurality of parallel processing paths. In a more detailed embodiment, the plurality of subsets of the genetic data may be in the form of binary alignment map (BAM) files at least at some point in the respective parallel processing paths. In a further detailed embodiment, the BAM files may include a first plurality of BAM files corresponding to read pairs in which both mates are mapped to the same data set, and at least one BAM file corresponding to read pairs in which both mates are mapped to different data sets. In a further detailed embodiment, the first plurality of BAM files may correspond to one or more segments of chromosomes with both mates mapped to the respective segments of chromosomes in each BAM file. In a further detailed embodiment, the total number of parallel processing paths may correspond to the number of processor cores respectively performing the parallel processing operations.

In an alternate detailed embodiment, the BAM files may include at least twenty-four BAM files, 22 corresponding to autosomal chromosomes and 2 corresponding to sex chromosomes. Alternatively, or additionally, the processing of a plurality of subsets of the genetic sequence data among the plurality of parallel processing paths may include a step of performing the parallel processing in a network cluster environment. Alternatively, or additionally, the processing of a plurality of subsets of the genetic sequence data among the plurality of parallel processing paths may be performed utilizing a cloud computing environment.

The performance of the workflow of the disclosure may be improved with the aid of various optimizations. Both software optimizations and hardware optimizations may be utilized.

System For Sequence Data Analysis

Another aspect of the present disclosure relates to a system for sequence data analysis. The system comprises (a) a cluster computing network, (b) a master computing unit for receiving sequencing data (reads) for a sequence device, (c) a plurality of computing nodes for parallel processing data in the cluster computing network, each node comprising a processor, and (d) a software container comprising software programs for sequence data analysis, in which each of the plurality of computing nodes has the same set of software programs installed thereon, and the multiple computing nodes are configured in the cluster computing network to execute the software programs.

In some embodiments, the software programs described herein comprise one or more software programs for read mapping.

In some embodiments, the software programs described herein comprise one or more software programs for variant calling.

In some embodiments, the software programs described herein comprise one or more software programs for annotation.

The reads described herein may be in the form of raw data generated from the sequence device or the sequence analyses, partially processed or processed data, and/or data files compatible with particular software programs. The input data files may take the form of FASTQ files, binary alignment files (BAM)*.bcl, *.vcf, and/or *.csv files. The output data files may be in formats that are compatible with available sequence data viewing, modification, annotation, and manipulation software. In certain embodiments, input data files from an initial DNA sequence are FASTQ files. In certain embodiments, input data files from read mapping are BAM files.

The performance of the systems of the disclosure may be improved with the aid of various optimizations. Both software optimizations and hardware optimizations may be utilized.

SeqsLab Platform

The present disclosure also provides a computational platform (which is referred herein as “SeqsLab”) that enables sequencing pipelines to be executed in parallel on a multi-node and/or multi-core compute infrastructure in a highly efficient manner. The platform adopts the Adaptive Data Parallelization (ADP) approach, and comprises a software container containing software programs for sequence data analysis.

The platform may fully automate the multiple steps required to go from raw sequencing reads to comprehensively annotated genetic variants. Through implementation of the computational platform, it has been found that testing of exemplary embodiments has shown a dramatic reduction in the analysis time.

It has been found that exemplary implementations of SeqsLab platform have achieved more than a ten-fold speedup in the time required to complete the analysis compared to a non-partitioning data workflow. Furthermore, SeqsLab platform has been designed with the flexibility to incorporate other analysis tools as they become available.

### EXAMPLES

In order that the invention described herein may be more fully understood, the following examples are set forth. It should be understood that these examples are for illustrative purposes only and are not to be construed as limiting this invention in any manner.

To test the above described parallel pipeline, sequence data was generated by the Illumina HiSeq 2500. The pipeline was also run on the publicly available data to test its performance on whole genome sequencing data.

### Example 1: Execution Time of GATK-HaplotypeCaller with and without Data Partition

Three outlined approaches were applied to whole genome sequencing data from a Bio-bank Sequencing Project. GATK 3.7 version of HaplotypeCaller was used for benchmarking. The execution time for GATK-HaplotypeCaller for (a) No Data Partitioning, (b) Data Partitioning by Chromosomes after read mapping, and (c) Data Partitioning by contiguous unmasked regions in the genome after read mapping are shown in Table 7. Compared to the execution time with no data partitioning, the execution time based on (b) data partitioning by chromosomes, and (c) data partitioning by contiguous unmasked regions is greatly reduced, respectively.

TABLE 7
Performance comparison based on the execution
time of GATK-HaplotypeCaller

| | | (b) | (c) |
|---|---|---|---|
| | (a) | Data | Data Partitioning |
| | No Data | Partitioning by | by contiguous |
| Strategy | Partitioning | Chromosomes | unmasked regions |
| Variant Calling | 1,603 min | 135 min | 46 min |
| (GATK | | | |
| HaplotypeCaller) | | | |

### Example 2: Execution Time of NGS Data Analysis with and without Data Partition

Three outlined approaches were applied to whole genome sequencing data from a Bio-bank Sequencing Project. Based on the GATK best practice, the results of the runtime from read mapping to variant calling with phasing information are shown in Table 8 illustrating three approaches of no data partition, data partitioning by chromosomes, and data partitioning by contiguous unmasked regions in the genome. Compared to the runtime by the no data partition method, the speed based on data partitioning by chromosomes is 5.0 times faster, and the speed based on data partitioning by contiguous unmasked regions is increased to 9.1 times faster.

TABLE 8
Benchmarking—CPU utilization on AWS r4.2x1arge (18 nodes)

| | | | Data |
|---|---|---|---|
| | | | Partitioning |
| | | Data | by |
| | | Partitioning | contiguous |
| | No Data | by | unmasked |
| Strategy | Partitioning | Chromosomes | regions |
| Data Partitioning (I) | — | 30 | 30 |
| Read Mapping | 440 | 65 | 65 |
| (BWA MEM) | | | |
| BAM Sorting and | 40 | 20 | 26 |
| Data Partitioning (II) | | | |
| Calling Preprocessing | 2,486 | 481 | 209 |
| (MarkDuplication, | | | |
| ReorderSam, | | | |
| AddOrReplaceReadGroups, | | | |
| BQSR, PrintReads) + | | | |
| Variant Calling | | | |
| (GATK HaplotypeCaller) + | | | |
| Haplotype Phasing | | | |
| (WhatsHAP) | | | |
| Total | 2,966 min | 596 min | 327 min |
| Speedup | 1 X | 5.0 X | 9.1 X |

The present disclosure provides a non-transitory storage medium having instructions therein, when executed, causing at least one processing unit to perform a method for facilitating optimization of a cluster computing network for sequencing data analysis using adaptive data parallelization, as exemplified in one of the embodiments. In an embodiment, a storage medium, such as non-transitory storage medium, stores computer-readable instructions (or program code), and the instructions are executed on at least one computing device, such that the at least one computing device carries out a method according to at least one of the embodiments. The method is illustrated by FIG. 4A, 4B, 5B, 6, 7, 9, 10, 11 or other and carried out according to one of the aforesaid embodiments or any combinations thereof, whenever appropriate. For instance, the program code comprises, for example, one or more programs or program modules, for use in carrying out the steps of the method based on at least one of embodiments or a combination thereof as illustrated by FIG. 4A, 4B, 5B, 6, 7, 9, 10, 11 or other and in any appropriate sequence. The embodiment of the storage medium includes, but is not limited to, optical information storage medium, magnetic information storage medium or memory (such as memory card, firmware, ROM or RAM). For instance, the computing device comprises a communication unit, processing unit and storage medium. The processing unit is electrically coupled to the communication unit and storage medium. The processing unit communicates with a communication network through the communication unit in a wireless or wired manner, so as to communicate with any other computing device, such as a terminal device. The processing unit comprises one or more processors. The computing device comprises any other device, such as a graphics processor, to perform computing. In an embodiment, the computing device can execute an operating system and is further implemented by one or more means of appropriate network and software technology, such as a server for network service, script engine, network application program or network application program interface (API).

While the present disclosure has been described by means of specific embodiments, numerous modifications and variations could be made thereto by those skilled in the art without departing from the scope and spirit of the present disclosure set forth in the claims.

What is claimed is:

1. A method for facilitating optimization of a cluster computing network for sequencing data analysis using adaptive data parallelization, the method comprising steps of:
(a) determining, by one or more processing units, a data parallelization configuration for a sequencing data analysis, based on sequencing data and a pipeline selection, wherein the data parallelization configuration includes partition indication data indicating at least one biological information unit according to which of the sequencing data is to be partitioned; and
(b) determining, by one or more processing units, at least one recommendation list for the sequencing data analysis, based on the data parallelization configuration and a computing resource list for the cluster computing network,
wherein the at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

2. The method according to claim 1, wherein in the step (a), the partition indication data indicates the at least one biological information unit according to which of the sequencing data is capable of being partitioned into a plurality of consecutive, non-overlapping, variable-length segments so as to retain biological meaning of the sequencing data.

3. The method according to claim 1, wherein the at least one biological information unit is at least one of chromosome, chromosome and discordant reads, centromere, or telomere.

4. The method according to claim 1, wherein the at least one biological information unit includes a contiguous unmasked region.

5. The method according to claim 1, wherein the at least one biological information unit includes a fixed length region.

6. The method according to claim 1, wherein the at least one biological information unit includes protein coding genes.

7. The method according to claim 1, wherein the at least one biological information unit includes genes.

8. The method according to claim 1, wherein the at least one biological information unit includes a user-defined biological unit.

9. The method according to claim 1, wherein in the step (b), each of the at least one recommendation list includes a plurality of computing resource entries, and a number of the computing resource entries of each of the at least one recommendation list is less than a number of computing resource entries included in the computing resource list.

10. The method according to claim 9, wherein the partition indication data indicates the at least one biological information unit according to which of the sequencing data is capable of being partitioned into a plurality of consecutive, non-overlapping, variable-length segments so as to retain biological meaning of the sequencing data.

11. The method according to claim 1, wherein the at least one recommendation list comprises a recommendation list for at least one portion of the sequencing data analysis, the recommendation list includes a plurality of computing resource entries indicating estimated processing times and corresponding estimated costs with respect to the at least one portion of the sequencing data analysis.

12. The method according to claim 1, wherein the at least one recommendation list comprises a plurality of recommendation lists for a plurality of portions of the sequencing data analysis, each of the recommendation lists includes a plurality of corresponding computing resource entries indicating estimated processing times and corresponding estimated costs with respect to a corresponding one of the plurality of portions of the sequencing data analysis.

13. The method according to claim 1, wherein the cluster computing network is an on-premises cluster computing network or a cloud computing network.

14. A non-transitory storage medium having instructions therein, when executed, causing at least one processing unit to perform a method for facilitating optimization of a cluster computing network for sequencing data analysis using adaptive data parallelization, according to claim 1.

15. A system for facilitating optimization of a cluster computing network for sequencing data analysis using adaptive data parallelization, the system comprising:
a memory; and
at least one processing unit coupled to the memory to perform operations including:
(a) determining a data parallelization configuration, based on sequencing data and a pipeline selection for a sequencing data analysis, wherein the data parallelization configuration includes partition indication data indicating at least one biological information unit according to which of the sequencing data is to be partitioned; and
(b) determining at least one recommendation list for the sequencing data analysis, based on the data parallelization configuration and a computing resource list for the cluster computing network,
wherein the at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.

16. The system according to claim 15, wherein in the operation (a), the partition indication data indicates the at least one biological information unit according to which of the sequencing data is capable of being partitioned into a plurality of consecutive, non-overlapping, variable-length segments so as to retain biological meaning of the sequencing data.

17. The system according to claim 15, wherein the at least one biological information unit is at least one of chromosome, chromosome and discordant reads, centromere, or telomere.

18. The system according to claim 15, wherein the at least one biological information unit includes a contiguous unmasked region.

19. The system according to claim 15, wherein the at least one biological information unit includes a fixed length region.

20. The system according to claim 15, wherein the at least one biological information unit includes protein coding genes.

21. The system according to claim 15, wherein the at least one biological information unit includes genes.

22. The system according to claim 15, wherein the at least one biological information unit includes a user-defined biological unit.

23. The system according to claim 15, wherein in the operation (b), each of the at least one recommendation list includes a plurality of computing resource entries, and a number of the computing resource entries of each of the recommendation list is less than a number of computing resource entries included in the computing resource list.

24. The system according to claim 15, wherein the partition indication data indicates the at least one biological information unit according to which of the sequencing data is capable of being partitioned into a plurality of consecutive, non-overlapping, variable-length segments so as to retain biological meaning of the sequencing data.

25. The system according to claim 15, wherein the at least one recommendation list comprises a recommendation list for at least one portion of the sequencing data analysis, the recommendation list includes a plurality of computing resource entries indicating estimated processing times and corresponding estimated costs with respect to the at least one portion of the sequencing data analysis.

26. The system according to claim 15, wherein the at least one recommendation list comprises a plurality of recommendation lists for a plurality of portions of the sequencing data analysis, each of the recommendation lists includes a plurality of corresponding computing resource entries indicating estimated processing times and corresponding estimated costs with respect to a corresponding one of the plurality of portions of the sequencing data analysis.

27. A method for facilitating optimization of a cluster computing network for sequencing data analysis using adaptive data parallelization, the method comprising steps of:
informing the cluster computing network to create a computing environment in the cluster computing network for a user; and
instructing the cluster computing network to deploy a software system for facilitating optimization for sequencing data analysis using adaptive data parallelization in the private computing environment for the user so that the private computing environment is capable of executing the software system to perform operations including:
(a) determining a data parallelization configuration for a sequencing data analysis, based on sequencing data and a pipeline selection, wherein the data parallelization configuration includes partition indication data indicating at least one biological information unit according to which of the sequencing data is to be partitioned; and
(b) determining at least one recommendation list for the sequencing data analysis, based on the data parallelization configuration and a computing resource list for the cluster computing network,
wherein the at least one recommendation list is for a computing device to produce at least one resource allocation selection from the at least one recommendation list so that the cluster computing network, in response to the at least one resource allocation selection, performs the sequencing data analysis on the sequencing data according to the at least one resource allocation selection and the data parallelization configuration.

28. A non-transitory storage medium having instructions therein, when executed, causing at least one processing unit to perform a method for facilitating optimization of a cluster computing network for sequencing data analysis using adaptive data parallelization, according to claim 27.

29. A system for facilitating optimization of a cluster computing network for sequencing data analysis using adaptive data parallelization, the system comprising:
a memory; and
at least one processing unit coupled to the memory to perform operations including:
informing the cluster computing network to create a private computing environment in the cluster computing network for a user; and
instructing the cluster computing network to install a software system for facilitating optimization for sequencing data analysis using adaptive data parallelization in the private computing environment for the user so that the private computing environment is capable of executing the software system to perform operations including:
(a) determining a data parallelization configuration, based on sequencing data and a pipeline selection for a sequencing analysis, wherein the data parallelization configuration includes partition indication data indicating at least one biological information unit according to which of the sequencing data is to be partitioned; and
(b) determining at least one recommendation list for the sequencing analysis, based on the data parallelization configuration and a computing resource list for the cluster computing network,
wherein the at least one recommendation list is for a computing device to produce at least one resource allocation selection from the at least one recommendation list so that the cluster computing network, in response to the at least one resource allocation selection, performs the sequencing data analysis on the sequencing data, according to the at least one resource allocation selection and the data parallelization configuration.
